(19) United States
(12) Patent Application Publication
(10) Pub. No.: US 2024/0383973 A1
(43) Pub. Date: Nov. 21, 2024

(54) METHODS FOR TREATING CALCITONIN GENE-RELATED PEPTIDE (CGRP) - EXPRESSING CANCERS

(71) Applicant: The Board of Regents of the University of Oklahoma, Norman, OK (US)

(72) Inventor: Maureen Cox, Edmond, OK (US)

(73) Assignee: The Board of Regents of the University of Oklahoma, Norman, OK (US)

(21) Appl. No.: 18/785,772

(22) Filed: Jul. 26, 2024

Related U.S. Application Data
(63) Continuation-in-part of application No. PCT/US2023/011466, filed on Jan. 24, 2023.
(60) Provisional application No. 63/304,484, filed on Jan. 28, 2022.

(51) Int. Cl. C07K 16/18; A61K 39/00; A61P 35/00; C07K 16/28

(52) U.S. Cl. CPC C07K 16/18; A61P 35/00; C07K 16/2818; A61K 2039/507; C07K 2317/76

(57) ABSTRACT

The present disclosure is directed to methods of cancer treatment, for example of solid tumors, in which CGRP is inhibited, and optionally is administered in tandem with checkpoint inhibitor immunotherapy (e.g., inhibition of Programmed cell death protein 1 (PD-1), PD-1 ligand, and/or Cytotoxic T-lymphocyte-Associated protein 4 (CTLA-4)) to bolster anti-tumor immunity and lead to tumor rejection. In one non-limiting embodiment, for example, the cancer treated is breast cancer. In this manner, CGRP inhibitors that target neurochemicals secreted by sensory nerves and are used as migraine treatments can be repurposed in anti-tumor therapy.

### CROSS-REFERENCE TO RELATED APPLICATIONS

The present application is a continuation-in-part of International application PCT/US2023/011466, filed Jan. 24, 2023, which claims the benefit of U.S. Provisional Patent Application Ser. No. 63/304,484, filed Jan. 28, 2022, the disclosures of which are hereby incorporated by reference in their entirety.

### STATEMENT REGARDING FEDERALLY SPONSORED RESEARCH OR DEVELOPMENT

This invention was made with government support under Grant Numbers GM103447 and GM103639 awarded by the National Institutes of Health. The government has certain rights in the invention.

### BACKGROUND

Neurons infiltrate solid tumors, however the impact of these nerves on tumor growth has only recently been explored. To date, work has focused on the impact of neural signaling directly on tumor cells, however immune cells also express receptors for a multitude of neurotransmitters, and signaling by different neurotransmitters can either enhance or inhibit immune function. In breast cancer, for example, increased presence of nerves in the tumor correlates with a poor prognosis in patients. Breast tissue contains both adrenergic and sensory nerve fibers, however it has been unknown what role each type of nerve plays in the context of tumorigenesis. Both tumors and immune cells can respond to neurotransmitters produced by these nerves. For example, depletion of sensory nerve fibers that express the capsacin receptor transient receptor potential cation channel subfamily V member 1 (TRPV1) results in greatly accelerated tumor growth, indicating these specific sensory nerve fibers mediate anti-tumor effects. A better understanding of the role and effects of the neurons and neurotransmitters in tumor tissues and tumor growth would be desirable, particularly regarding how such an understanding could lead to improved cancer treatments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows that depletion of TRPV1⁺ sensory nerves accelerates growth of Py230 tumors. B6 mice (squares) or Rag1^{-/-} mice (circles) were either depleted of TRPV1⁺ nerves by RTX treatment (white) or given vehicle control (black) 2 weeks prior to tumor transplantation. Tumor mass measured 60 days after transplant. Symbols represent individual mice, mean+/−S.D. Composite of 3-5 experimental cohorts. *p<0.05; **p<0.01.

FIGS. 2A to 2C shows that inhibition of CGRP signaling inhibits tumor growth and promotes anti-tumor immunity. B6 mice were transplanted with Py230 tumor cells in the 4^{th} mammary fat pad. Beginning on Day 28 post-transplant, mice were either injected with the CGRP inhibitor BIBN4096 in DMSO (white) or DMSO alone (black). 63 days post-transplant, tumors were isolated and tumor mass (FIG. 2A) determined. Tumors were digested and the ratio of activated CD44^{hi} CD43⁺CD8 T cells to FoxP3⁺ Tregs determined (FIG. 2B). PD-1 expression on tumor-isolated CD8+ T cells was measured by flow cytometry (FIG. 2C). Symbols represent individual mice, composite of two experiments. *p<0.05, **p<0.01.

FIG. 3 shows the correlation of SP and CGRP receptor expression with breast cancer survival. The Human Protein Atlas was interrogated for TACR1 (SP receptor) and CALCRL (CGRP receptor) expression in breast cancer. High expression (pink) and low expression (blue) is contrasted with survival over time in breast cancer patients. The number of patients is indicated on graph.

FIG. 4 shows the effects of an anti-CGRP/anti-PD-1 combination treatment on tumor volume in mice transplanted with a PY230 murine mammary tumor. The anti-PD-1 compound is a rat IgG2a anti-mouse PD-1 antibody, clone RMP1-14, obtained from Bio X Cell. The CGRP inhibitor is olcegepant. The isotype control is a rat IgG2a, clone 2A3, raised against human CD25, obtained from Bio X Cell.

FIG. 5 shows the effects of the anti-CGRP/anti-PD-1 combination treatment on tumor regression in the mice of FIG. 4.

FIG. 6 shows the effects of the anti-CGRP/anti-PD-1 combination treatment of FIG. 4 on the survival of mice transplanted with PY230 murine mammary tumors.

FIG. 7 shows the effects of the anti-CGRP/anti-PD-1 combination treatment of FIG. 4 on tumor mass of mice transplanted with PY230 murine mammary tumors.

FIG. 8 shows the effects of the anti-CGRP/anti-PD-1 combination treatment of FIG. 4 on tumor metastases in lungs in mice transplanted with PY230 murine mammary tumors.

FIGS. 9A to 9C shows that breast cancer produces calcitonin gene related peptide (CGRP) in the absence of sensory nerves. FIG. 9A. Immunohistochemical staining for sensory nerve (top panel—arrow) and CGRP (bottom panel) in sensory nerve depleted breast cancer samples and their respective controls day 60 post-transplant. FIG. 9B. CGRP concentration (pg/μL) in Py230 cell line and mammary epithelial cells (MECs) as control prior to transplant. FIG. 9C. CGRP concentration (pg/μL) normalized to mg of tumor tissue at days 15, 30, 45 and 60 post-transplant.

FIGS. 10A and 10B shows that BIBN4096, CGRP antagonist, stops growth of breast cancer and decreases tumor mass. B6 mice were transplanted with Py230 triple negative breast cancer cell line, tumors were measured 3×/week for 63 days. BIBN4096 or DMSO vehicle control (mock) were administered 1×/week starting on day 28 over a period of 4 weeks. Tumor masses were measured at day 63. FIG. 10A. Tumor growth curves of BIBN4096 treated B6 mice and mock treated controls. FIG. 10B. Tumor masses of BIBN4096 treated mice and mock treated controls. (n=10/group), Mann-Whitney Test, *p<0.05.

FIGS. 11A to 11D shows that BIBN4096, CGRP antagonist enhances CD8+ T cell activation and tumor infiltrating B cells. B6 mice were transplanted with Py230 triple negative breast cancer cell line, tumors were measured 3×/week for 63 days. BIBN4096 or DMSO vehicle control (mock) were administered 1×/week starting on day 28 over a period of 4 weeks. Tumor was passed through a 70 m cell strainer and single cell suspension was stained for flowcytometric analysis. FIG. 11A. BIBN4096 treated mice have a higher ratio of activated CD8⁺ T cells to regulatory T cells (T reg) compared to the mock treated controls. FIG. 11B. Activated CD8+ T cells have an increased expression of the inhibitory receptor programmed death 1 (PD-1) in BIBN4096 treated mice compared to their controls. FIG. 11C. BIBN4096 treated mice had an increased infiltration of tumor infiltrating B cells. FIG. 11D. Tumor infiltrating T cell subtypes at day 63 in BIBN4096 and mock treated controls. (n=15 group) Mann-Whitney Test, *p<0.05, **p<0.01, ****p<0.0001, n.s. not significant.

FIG. 12A shows a Kaplan Meier survival graph show that mice cotreated with BIBN4096 and PD-1 antibody have better survival compared to their respective controls. B6 mice were transplanted with Py230 triple negative breast cancer cell line, tumors were measured 3×/week for 126 days to overlook survival. BIBN4096 or vehicle control were administered 1×/week starting on day 28 for 4 weeks. PD-1 monoclonal antibody and control were administered every other day starting on day 28 for 4 weeks. C. Tumor masses (g) of mice cotreated with BIBN4096 and PD-1 antibody and respective controls D. Number of mice that presented with metastasis in the four groups. (n=5/group).

FIG. 12B shows tumor masses (g) of mice cotreated with BIBN4096 and PD-1 antibody and respective controls. Cotreatment substantially reduces tumor mass. B6 mice were transplanted with Py230 triple negative breast cancer cell line, tumors were measured 3×/week for 126 days to overlook survival. BIBN4096 or vehicle control were administered 1×/week starting on day 28 for 4 weeks. PD-1 monoclonal antibody and control were administered every other day starting on day 28 for 4 weeks.

FIG. 12C shows the number of mice that presented with metastasis after being cotreated with BIBN4096 and PD-1 antibody, n=5/group. None of the cotreated mice had detectable metastases. B6 mice were transplanted with Py230 triple negative breast cancer cell line, tumors were measured 3×/week for 126 days to overlook survival. BIBN4096 or vehicle control were administered 1×/week starting on day 28 for 4 weeks. PD-1 monoclonal antibody and control were administered every other day starting on day 28 for 4 weeks.

The following abbreviations may be used herein.
ADC: antibody-drug conjugate,
BIBN4096: Olcegepant,
CALCRL: calcitonin receptor-like receptor,
CD279: Cluster of differentiation 279 protein,
CDR: Complementarity determining regions,
CGRP: Calcitonin gene-related peptide,
CTLA-4: Cytotoxic T-lymphocyte-Associated protein 4,
PD-1: Programmed cell death protein 1,
PD-L1: Programmed cell death protein 1,
RAMP1: receptor activity-modifying protein 1,
RTX: resiniferatoxin, and
TRPV1: transient receptor potential cation channel subfamily V member 1.

### DETAILED DESCRIPTION

It has been discovered herein that inhibition of the sensory nerve-produced neuropeptide calcitonin gene-related peptide (CGRP) inhibits tumor growth and improves immune presence in tumors, particularly in solid cancerous tumors. Inhibition of CGRP stalled tumor growth and resulted in enhanced infiltration by activated CD8+ T cells. This is highly significant, as recent immunotherapeutic treatments that have revolutionized treatment of melanoma have largely failed as treatments for certain cancers, such as breast cancer.

In certain embodiments, the present disclosure is directed to methods of cancer treatment, for example of solid tumors, in which CGRP is inhibited in tandem with checkpoint inhibitor immunotherapy (e.g., inhibition of Programmed cell death protein 1 (PD-1), PD-1 ligand (PD-1L), and/or Cytotoxic T-lymphocyte-Associated protein 4 (CTLA-4)) to bolster anti-tumor immunity and lead to tumor rejection. In one non-limiting embodiment, for example, the cancer treated is a breast cancer, such as triple negative breast cancer. In this manner, CGRP inhibitors that target neurochemicals secreted by sensory nerves and are used as migraine treatments can be repurposed in anti-tumor therapy.

### CGRP

CGRP is a 37 amino acid neuropeptide of the peptide family that includes calcitonin, adrenomedullin and amylin. In humans, two forms of CGRP (alpha-CGRP and beta-CGRP) exist and have similar activities. They vary by three amino acids and exhibit differential distribution. At least two CGRP receptor subtypes may also account for differential activities. The CGRP receptor is located within pain-signaling pathways, intracranial arteries and mast cells and its activation is thought to play a causal role in migraine pathophysiology. For example, research and clinical studies have shown that serum levels of CGRP are elevated during migraine attacks, infusion of intravenous CGRP produces persistent pain in migraine sufferers and non-migraine sufferers, and treatment with anti-migraine drugs normalizes CGRP activity. CGRP mediates its effects through a heteromeric receptor composed of a G protein-coupled receptor called calcitonin receptor-like receptor (CALCRL) and a receptor activity-modifying protein 1 (RAMP1). As used herein, “CGRP receptor signaling” refers to the receptor/ligand binding and activation of downstream targets. Inhibition of “CGRP receptor signaling” can be achieved by inhibition, e.g., of CGRP binding to CALCRL, of CALCRL, or of RAMP1. Alternatively, inhibition of “CGRP receptor signaling” can be achieved direct inhibition of downstream factors which are activated by CGRP receptor signaling, or direct activation of downstream targets which are inhibited by CGRP receptor signaling.

### PD-1 and PD-L1

PD-1 is 290 amino acid protein also known as CD279. The amino acid sequence of the human full-length PD-1 protein is set forth, for example, in GenBank Accession Number NP_005009.2. The term “PD-L1”, also known as CD274, refers to a ligand of the PD-1 receptor having the sequence set forth, for example, in GenBank Accession Number NP_054862.1 PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). PD-L1 is also expressed on a wide variety of tumors, virally-infected cells, and autoimmune tissue. The programmed cell death 1 (PD-1/PD-L1) pathway acts as a checkpoint to limit T-cell-mediated immune responses. A second PD-1 ligand, PD-L2, can also engage the PD-1 receptor and induce PD-1 signaling and reversible inhibition of T-cell activation and proliferation. When PD-1 ligands are on the surface of cancer cells or neighboring cells, these ligands bind to PD-1 receptor positive immune effector cells and utilize the PD-1 pathway to evade an immune response.

### CTLA-4

CTLA-4 is a 223 amino acid protein, also known as CD152. CTLA-4 is a member of the immunoglobulin superfamily that is expressed exclusively on T-cells. CTLA-4 acts to inhibit T cell activation and is reported to inhibit helper T cell activity and enhance regulatory T cell immunosuppressive activity. CTLA-4 is upregulated after T cell activation and functions as an immune checkpoint, downregulating the T cell activation and immune activity.

It is well established that the interaction between PD-1 and PD-1L can act as an immune checkpoint, which can lead to negative effects such as, a decrease in tumor infiltrating lymphocytes, a decrease in T-cell receptor mediated proliferation, and/or immune-evasion by cancerous cells. PD-1 is an inhibitory member of the extended CD28/CTLA-4 family of T cell regulators. PD-1 is expressed on activated B cells, T cells, and monocytes. PD-L1 and PD-L2, the two forms of PD-1 ligand, have been shown to downregulate T cell activation upon binding to PD-1. Both PD-L1 and PD-L2 are homologs that bind to PD-1. Immune suppression can be reversed by inhibiting the local interaction of PD-1 with PD-L1 or PD-L2. PD-L1 is particularly abundant in a variety of human cancers.

Monoclonal antibodies as PD-1 antagonists have been developed in recent years for use in therapy, more precisely for treating various diseases, including cancer and infectious diseases. Any one of such anti-PD-1- or anti-PD-L1 antibodies, or binding fragments thereof, can be used according to the present disclosure. It has been surprisingly discovered herein that treatment with a combination of a CGRP inhibitor and a PD-1 antagonist, as defined herein, can lead to a significantly stronger reduction, or even shrinkage, in tumor volume as compared to single treatment with a CGRP inhibitor or a PD-1 antagonist. The PD-1 antagonist may be an inhibitor of PD-1 or an inhibitor of PD-L1. The PD-1 antagonist may be an anti-PD-1-antibody or an anti-PD-L1-antibody or an anti-PD-L2 antibody, including a humanized or fully human anti-PD-1 antibody or a humanized or fully human anti-PD-L1 antibody, or fragments thereof. Any one of these antibodies may be a recombinant human antibody, or a binding fragment thereof. In various alternative embodiments of the present disclosure, the CGRP inhibitor and a checkpoint inhibitor (a PD-1 antagonist and/or a CTLA-4 inhibitor) may be administered in simultaneous, concurrent, sequential, successive, alternate or separate steps. The CGRP inhibitor and checkpoint inhibitor may be administered either as part of the same pharmaceutical composition/dosage form or, in separate pharmaceutical compositions and/or dosage forms.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, published patent applications, and non-patent publications including published articles mentioned in the specification or referenced in any portion of this application, including but not limited to, U.S. Provisional Patent Application Ser. No. 63/304,484, filed Jan. 28, 2022, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

The term “active agent” as used herein is intended to refer to a substance which possesses a biological activity relevant to the present disclosure, and particularly refers to therapeutic and diagnostic substances which may be used in methods described in the present disclosure. “Biologically active” refers to the ability of a substance to modify the physiological system of a cell, tissue, or organism without reference to how the substance has its physiological effects.

The term “small molecule” as used herein refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals or subjects within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent, or combination of active agents, which is sufficient to exhibit a detectable therapeutic or treatment effect in a subject without excessive adverse side effects (such as substantial toxicity, irritation, and allergic response) commensurate with a reasonable benefit/risk ratio when used in the manner of the present disclosure. The effective amount for a subject will depend upon the subject's type, size, and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein. An effective amount can be sufficient to reduce and/or ameliorate the progression, development, recurrence, severity and/or duration of a given disease, disorder or condition and/or a symptom related thereto, or can be sufficient to reduce the level of activity or binding of a polypeptide (e.g., CGRP, CGRP receptor, PD-L1, PD-1, or CTLA-4).

The term “ameliorate” means a detectable or measurable improvement in a subject's condition, disease, or symptom thereof. A detectable or measurable improvement includes a subjective or objective decrease, reduction, inhibition, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a disease or condition, or consequences of the disease or condition in a subject.

The terms “inhibition”, “inhibit”, “inhibiting” refer to a reduction in the activity, binding, or expression of a polypeptide or reduction or amelioration of a disease, disorder, or condition or a symptom thereof. Inhibiting as used here can include partially or totally blocking stimulation, decreasing, preventing, or delaying activation or binding, or inactivating, desensitizing, or down-regulating protein or enzyme activity or binding.

A decrease or reduction in worsening, such as stabilizing the condition or disease, is also a successful treatment outcome. A therapeutic benefit therefore need not be complete ablation or reversal of the disease or condition, or any one, most, or all adverse symptoms, complications, consequences, or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

The term “regimen” refers to a protocol for dosing and timing the administration of one or more therapies (e.g., combinations described herein or another active agent such as an anti-cancer agent described herein) for treating a disease, disorder, or condition described herein. A regimen can include periods of active administration and periods of rest as known in the art. Active administration periods include administration of combinations and compositions described herein and the duration of time of efficacy of such combinations and compositions. Rest periods of regimens described herein include a period of time in which no compound is actively administered, and in certain instances, includes time periods where the efficacy of such compounds can be minimal. Combination of active administration and rest in regimens described herein can increase the efficacy and/or duration of administration of the combinations and compositions described herein.

The terms “therapies” and “therapy” refer to any protocol(s), method(s), and/or agent(s) that can be used in the prevention, treatment, management, and/or amelioration of a disease, disorder, or condition or one or more symptoms thereof. In certain instances the term refers to active agents such as an anti-cancer agent described herein. The terms “therapy” and “therapy” can refer to anti-viral therapy, anti-bacterial therapy, anti-fungal therapy, anti-cancer therapy, biological therapy, supportive therapy, and/or other therapies useful in treatment, management, prevention, or amelioration of a disease, disorder, or condition or one or more symptoms thereof known to one skilled in the art, for example, a medical professional such as a physician.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The term “enhance” refers to an increase or improvement in the function or activity of a protein or cell after administration or contacting with a combination described herein compared to the protein or cell prior to such administration or contact.

The active agents of the present disclosure can be combined into formulations or treatments that are synergistic. As used herein the terms “synergism,” “synergistic,” or “synergistic effect” refers to a therapeutic effect or result that is greater than the additive effects of each active agent used individually. Presence or absence of a synergistic effect for a particular combination of treatment substances can be quantified by using the Combination Index (CI) (e.g., Chou, Pharmacol Rev, 2006. 58(3): 621-81), wherein CI values lower than 1 indicate synergy and values greater than 1 imply antagonism. Combinations of the inhibitors and antagonists of the present disclosure can be tested in vitro for synergistic cell growth inhibition using standard cell lines for particular cancers, or in vivo using standard animal cancer models. A synergistic effect of a combination described herein can permit, in some embodiments, the use of lower dosages of one or more of the components of the combination (e.g., a CGRP inhibitor, and a PD-1 inhibitor and/or a CTLA-4 inhibitor). A synergistic effect can also permit, in some embodiments, less frequent administration of at least one of the administered active agents. Such lower dosages and reduced frequency of administration can reduce the toxicity associated with the administration of at least one of the therapies to a subject without reducing the efficacy of the treatment.

The term “administering” refers to the act of delivering a combination or composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arteriole, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition).

The term “coadministration” refers to administration of two or more active agents (e.g., a combination described herein and another active agent such as an anti-cancer agent described herein). The timing of coadministration depends in part of the combination and compositions administered and can include administration at the same time, just prior to, or just after the administration of one or more additional therapies, for example cancer therapies such as chemotherapy, hormonal therapy, radiotherapy, or immunotherapy. The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compounds described herein can be used in combination with one another, with other active agents known to be useful in treating cancer.

As used herein, “inhibition of CGRP” refers to the inhibition of CGRP expression, activity, CGRP release, or CGRP receptor signaling.

As used herein, “inhibition of PD-1” refers to the inhibition of PD-1 or PD-1 ligand expression, activity, or PD-1 receptor signaling.

As used herein, “inhibition of CTLA-4” refers to the inhibition of CTLA-4 expression, activity, release, or CTLA-4 receptor signaling.

The term “antibody” as used herein can refer to both intact “full length” antibodies as well as to CGRP-binding fragments, CGRP receptor-binding fragments, PD-1-binding fragments, PD-L1-binding fragments, CTLA-4-binding fragments, and CTLA-4 receptor-binding fragments thereof. The afore-mentioned binding fragments may also be referred to herein as antibody fragments, antigen binding compounds, antigen binding fragments, antigen binding portions, binding fragments, or binding portions. As used herein, the term “antibody” includes, but is not limited to, synthetic antibodies, monoclonal antibodies, recombinantly produced antibodies, intrabodies, multispecific antibodies (e.g., bispecific and trispecific antibodies), human antibodies, humanized antibodies, chimeric antibodies, recombinant single chain polypeptide molecules in which light and heavy chain variable regions are connected by a peptide linker, i.e., single-chain Fv (scFv) fragments, bivalent scFv (bi-scFv), trivalent scFv (tri-scFv), Fab fragments, Fab′ fragments, F(ab′) fragments, F(ab′)₂ fragments, F(ab)₂ fragments, disulfide-linked Fvs (sdFv) (including bi-specific sdFvs), and anti-idiotypic (anti-Id) antibodies, dAb fragments, nanobodies, diabodies, triabodies, tetrabodies, linear antibodies, isolated CDRs, and epitope-binding fragments of any of the above. Regardless of structure, an antibody fragment refers to an isolated portion of the antibody that binds to the same antigen that is recognized by the intact antibody. For example, an anti-CGRP antibody fragment binds with an epitope of CGRP. Antibody fragments can be produced by recombinant DNA techniques or by enzymatic or chemical separation of intact immunoglobulins.

Where reference is made anywhere herein to a particular antibody for use against CGRP, PD-1, PD-1L, and/or CTLA-4, including in the claims, such reference is also intended to include anti-CGRP binding fragments thereof, anti-PD-1 binding fragments thereof, anti-PD-1L binding fragments thereof, and/or anti-CTLA-4 binding fragments thereof, respectively.

Where reference is made anywhere herein to a particular antibody, protein, oligopeptide, or peptide, for use against CGRP, PD-1, PD-1L, and/or CTLA-4, including in the claims, such reference is also intended to include biosimilars of the particular antibody, protein, oligopeptide, or peptide, or binding fragment thereof.

The term “biosimilar” as used herein refers to a biological drug, such as an antibody, or binding fragment of an antibody, that is very much like another biological drug (the reference drug) that is already approved by the USFDA. The biosimilar may be made in different ways from, but has no clinically-meaningful differences in structure and activity from, the reference drug, and is used in the same way for the same purpose as the reference drug. For example, the biosimilar may have differences in the amino acid sequence or differences in post-translations modifications such as slight differences in glycosylation.

The term “target antigen” in at least certain embodiments refers to CGRP, CGRP receptor, PD-1, PD-1L, CTLA-4, and CTLA-4 receptor, and epitopes and fragments thereof, which may have biological activity.

The antibodies of several embodiments provided herein may be monospecific, bispecific, trispecific, or of greater multispecificity, such as multispecific antibodies formed from antibody fragments. The term “antibody” also includes a diabody (homodimeric Fv fragment) or a minibody (V_{L}-V_{H}-C_{H3}), a bispecific antibody, or the like. A bispecific or bifunctional antibody is an artificial hybrid antibody having two different heavy/light chain pairs and two different binding sites. Multispecific antibodies may be specific for different epitopes of a polypeptide or may be specific for both a polypeptide as well as for a heterologous epitope, such as a heterologous polypeptide or solid support material. Single chain antibodies produced by joining antibody fragments using recombinant methods, or a synthetic linker, are also encompassed by the present disclosure (e.g., see, for example, published PCT application Nos. WO1993/17715; WO1992/08802; WO1991/00360; and WO1992/05793; and U.S. Pat. Nos. 4,474,893; 4,714,681; 4,925,648; 5,573,920; and 5,601,819).

Natural antibodies are capable of binding to only one epitope species (i.e., they are “mono-specific”), although they may be able to bind multiple copies of that species (i.e., they may exhibit bi-valency or multi-valency). As noted above, the functionality of antibodies can be enhanced by generating multispecific antibody-based molecules that can simultaneously bind two separate and distinct antigens (or different epitopes of the same antigen) and/or by generating antibody-based molecule having higher valency (i.e., more than two binding sites) for the same epitope and/or antigen. Therefore, the antibodies used in the methods of the present disclosure may be monospecific, bispecific, trispecific, or of greater multispecificity, such as multispecific antibodies formed from antibody fragments. The term “antibody” also includes a diabody (homodimeric Fv fragment) or a minibody (V_{L}-V_{H}-C_{H3}), a bispecific antibody, or the like. A bispecific or bifunctional antibody is an artificial hybrid antibody having two different heavy/light chain pairs and two different binding sites. Multispecific antibodies may be specific for different epitopes of a polypeptide or may be specific for both a polypeptide as well as for a heterologous epitope, such as a heterologous polypeptide or solid support material. Single chain antibodies produced by joining antibody fragments using recombinant methods, or a synthetic linker, are also encompassed by the present disclosure (e.g., see, for example, published PCT applications Nos. WO2014/022540, WO2013/003652, WO2013/006544, WO2013/070565, WO2013/174873, WO2013/163427, WO2013/119903, WO2012/156430, WO2012/009544, WO2012/162583, WO2012/156430, WO2011/086091, WO2011/133886, WO2010/136172, WO2010/108127, WO2010/028797, WO2010028796, WO2010/028795, WO2009/132876, WO2009/018386, WO2008/027236, WO2008/003116, WO2008/003103, WO2007/146968, WO2007/046893, WO2007/075270, WO2006/107786, WO2006/072152, WO2006/107617, WO2005/070966, WO2002/020039, WO2000/018806; WO1999/042597, WO1998/006749, WO1998/003670, WO1998/002463, WO1993/17715; WO1992/08802; WO1992/022583, WO1991/003493, WO1991/00360; and WO1992/05793; U.S. Pat. Nos. 4,474,893; 4,714,681; 4,925,648; 5,573,920; 5,601,819; 6,171,586; 6,551,592; 6,994,853; 7,695,936; and 8,277,806; and U.S. Patent Publication Nos. 2010/0291112, 2008/0057054, and 2007/0196363.

The term “monoclonal antibody” as used herein refers to an antibody obtained from a population of substantially homogeneous antibodies, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method. For example, monoclonal antibodies can be made by the hybridoma method first described by Kohler et al. (Nature, 256:495 (1975)), or may be made by recombinant DNA methods (see, for example, U.S. Pat. No. 4,816,567).

An “isolated” antibody refers to an antibody that has been identified and separated and/or recovered from components of its natural environment and/or an antibody that is recombinantly produced. A “purified antibody” is an antibody that is typically at least 50% w/w pure of interfering proteins and other contaminants arising from its production or purification but does not exclude the possibility that the monoclonal antibody is combined with an excess of pharmaceutical acceptable carrier(s) or other vehicle(s) intended to facilitate its use. Interfering proteins and other contaminants can include, for example, cellular components of the cells from which an antibody is isolated or recombinantly produced. Sometimes monoclonal antibodies are at least 60%, at least 70%, at least 80%, at least 90%, at least 95%, or at least 99% w/w pure of interfering proteins and contaminants from production or purification. The antibodies and antibody-derived compounds described herein can be provided in isolated and/or purified form.

In at least certain embodiments of the present disclosure, the term “therapeutic agent” refers to an active agent comprising an antibody and/or antibody-derived compound or other compound as described herein that has a therapeutic activity.

A “diagnostic agent” is a substance that is useful in diagnosing a disease. Useful diagnostic agents of the present disclosure may include antibodies and antibody-derived compounds described herein, and may further comprise by linkage or other association radioisotopes, dyes, contrast agents, fluorescent compounds or molecules, and enhancing agents (e.g., paramagnetic ions).

An “immunoconjugate” or “antibody-drug conjugate” (ADC) is a conjugate of an antibody or antibody-derived compound with an atom, molecule, or a higher-ordered structure (e.g., with a liposome), a therapeutic agent, or a diagnostic agent.

As used herein, the term “antibody fusion protein” is a recombinantly produced antigen-binding molecule in which an antibody or antibody fragment is linked to another protein or peptide, such as the same or different antibody or antibody fragment. The fusion protein may comprise a single antibody component, a multivalent or multispecific combination of different antibody components, or multiple copies of the same antibody component, or other component described elsewhere herein. An example of a multivalent or multispecific antibody is a bispecific antibody which is able to target two different proteins or receptors. For example, a bispecific antibody may be able to target both PD-1 and CTLA-4, or both PD-1L and CTLA-4, or both PD-1 and PD-1L. The fusion protein may additionally comprise an antibody or an antibody fragment and a therapeutic agent.

The basic structural unit of an antibody is a tetramer of subunits. Each tetramer includes two identical pairs of polypeptide chains, each pair having one “light” chain (about 25 kDa) and one “heavy” chain (about 50-70 kDa). The amino-terminal portion of each chain includes a variable region of about 100 to 120 or more amino acids, including portions called complementarity determining regions (CDRs) as described below, which are primarily responsible for antigen recognition. This variable region is initially expressed linked to a cleavable signal peptide. The variable region without the signal peptide is sometimes referred to as a mature variable region. Thus, for example, a “light chain mature variable region” means a light chain variable region without the light chain signal peptide. The carboxy-terminal portion of each chain defines a constant region primarily responsible for effector function. A CDR is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest.

Light chains are classified as either kappa or lambda. Heavy chains are classified as gamma, mu, alpha, delta, or epsilon, and define the antibody's isotype as IgG, IgM, IgA, IgD, and IgE, respectively. Within light and heavy chains, the variable and constant regions are joined by a “J” region of about 12 or more amino acids, with the heavy chain also including a “D” region of about 10 or more amino acids.

The mature variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, which as noted above are known as CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3, and FR4.

The assignment of amino acids to each domain (FR1, CDR1, FR2, CDR2, FR3, CDR3, and FR4) is done in accordance with the protocols of the IMGT, e.g., see Ehrenmann, F., Kaas, Q. and Lefranc, M.-P., “IMGT/3Dstructure-DB and IMGT/DomainGapAlign: a database and tool for immunoglobulins or antibodies, T cell receptors, MHC, IgSF and MhcSF,” Nucl. Acids Res., 38(S1):D301-D307 (2010). DOI:10.1093/nar/kgp946. PMID:19900967, and Ehrenmann, F. and Lefranc, M.-P. Cold Spring Harb. Protocols, 2011(6):737-749. DOI:10.1101/pdb.prot5636. PMID:21632775. Using these IMGT protocols, CDR sequences can be determined from the amino acid sequences of each antibody.

In other embodiments, the assignment of amino acids to each domain may be done in accordance with the protocols of Kabat (Sequences of Proteins of Immunological Interest, National Institutes of Health, Bethesda, Md., 1987 and 1991), or Chothia & Lesk (J. Mol. Biol. 196:901-917 (1987); Chothia et al., Nature, 342:878-883 (1989)). Kabat also provides a widely used numbering convention (Kabat numbering) in which corresponding residues between different heavy chains or between different light chains are assigned the same number. Using these protocols, CDR sequences can be determined from the amino acid sequences of each antibody.

In certain non-limiting embodiments, the presently disclosed antibodies, or antigen-binding portions thereof, contain at least one heavy chain variable region and/or at least one light chain variable region. The heavy chain variable region (or light chain variable region) may contain three CDRs and four FRs, arranged from amino-terminus to carboxy-terminus in the following order: FR1, CDR1, FR2, CDR2, FR3, CDR3, FR4.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., “Epitope Mapping Protocols,” in Methods in Molecular Biology, Vol. 66, Glenn E. Morris, Ed. (1996).

Also within the scope of the present disclosure are antibodies or antibody-derived compounds thereof in which specific amino acids have been substituted, deleted, or added. These alternations do not have a substantial effect on the peptide's biological properties, such as (but not limited to) binding activity. For example, antibodies may have amino acid substitutions in the framework region, such as to improve binding to the antigen. In another example, a selected, small number of acceptor framework residues can be replaced by the corresponding donor amino acids. The donor framework can be a mature or germline human antibody framework sequence or a consensus sequence.

For purposes of classifying amino acids substitutions as conservative or nonconservative, amino acids are grouped in one non-limiting embodiment as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same group. Non-conservative substitutions constitute exchanging a member of one of these groups for a member of another.

Tables of conservative amino acid substitutions have been constructed and are known in the art. In other embodiments, examples of interchangeable amino acids include, but are not limited to, the following: arginine and lysine; glutamate and aspartate; serine and threonine; glutamine and asparagine; and valine, leucine, and isoleucine. In other non-limiting embodiments, the following substitutions can be made: Ala (A) by leu, ile, or val; Arg (R) by gln, asn, or lys; Asn (N) by his, asp, lys, arg, or gln; Asp (D) by asn or glu; Cys (C) by ala or ser; Gln (Q) by glu or asn; Glu (E) by gln or asp; Gly (G) by ala; His (H) by asn, gln, lys, or arg; Ile (I) by val, met, ala, phe, or leu; Leu (L) by val, met, ala, phe, or ile; Lys (K) by gln, asn, or arg; Met (M) by phe, ile, or leu; Phe (F) by leu, val, ile, ala, or tyr; Pro (P) by ala; Ser (S) by thr; Thr (T) by ser; Trp (W) by phe or tyr; Tyr (Y) by trp, phe, thr, or ser; and Val (V) by ile, leu, met, phe, or ala.

Other considerations for amino acid substitutions include whether or not the residue is located in the interior of a protein or is solvent- (i.e., externally) exposed. For interior residues, conservative substitutions include for example: Asp and Asn; Ser and Thr; Ser and Ala; Thr and Ala; Ala and Gly; Ile and Val; Val and Leu; Leu and Ile; Leu and Met; Phe and Tyr; and Tyr and Trp. For solvent-exposed residues, conservative substitutions include for example: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; and Phe and Tyr.

Percentage sequence identities can be determined with antibody sequences maximally aligned by the Kabat numbering convention. After alignment, if a particular antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises an antibody may contain the antibody alone or in combination with other ingredients. The phrase “pharmaceutically acceptable salt” refers to pharmaceutically acceptable organic or inorganic salts of a presently-disclosed antibody, or binding fragment, or conjugate thereof, or agent administered with a presently-disclosed antibody or fragment or conjugate thereof. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as (but not limited to) an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterions.

A chimeric antibody is a molecule in which different portions are derived from different animal species. For example, an antibody may contain a variable region derived from a murine mAb and a human immunoglobulin constant region. Chimeric antibodies can be produced by recombinant DNA techniques. For example, a gene encoding a murine (or other species) monoclonal antibody molecule is digested with restriction enzymes to remove the region encoding the murine Fc, and the equivalent portion of a gene encoding a human Fc constant region is substituted. Chimeric antibodies can also be created by recombinant DNA techniques where DNA encoding murine variable regions can be ligated to DNA encoding the human constant regions, e.g., see published PCT applications Nos. WO 87/002671 and WO 86/01533, and U.S. Pat. No. 4,816,567.

A chimeric antibody is a recombinant protein that contains the variable domains including the CDRs of an antibody derived from one species, for example a rodent or rabbit antibody, while the constant domains of the antibody molecule are generally derived from those of a human antibody. For veterinary applications, the constant domains of the chimeric antibody may be derived from that of other species, such as but not limited to, a cat, dog, or horse.

A chimeric antibody can be humanized by replacing the sequences of, for example, a murine FR in the variable domains of the chimeric antibody with one or more different human FR sequences. Specifically, mouse CDRs are transferred from heavy and light variable chains of the mouse immunoglobulin into the corresponding variable domains of a human antibody. As simply transferring mouse CDRs into human FRs may result in a reduction of antibody affinity, additional modifications might be required in order to restore the original affinity of the murine antibody. This can be accomplished by the replacement of one or more human residues in the FR regions with their murine counterparts to obtain an antibody with enhanced binding affinity to target antigens described herein. Techniques for producing humanized antibodies are weel known and are disclosed, for example, by Jones et al. (Nature, 321: 522 (1986)), Riechmann et al. (Nature, 332: 323 (1988)), Verhoeyen et al. (Science, 239: 1534 (1988)), Carter et al. (Proc. Nat'l Acad. Sci. USA, 89: 4285 (1992)), Sandhu (Crit. Rev. Biotech. 12:437 (1992)), and Singer et al. (J. Immun. 150: 2844 (1993)).

As noted, an antibody's light or heavy chain variable region comprises an FR having three different CDRs. In one non-limiting embodiment, humanized antibodies are antibody molecules from non-human species having one, two, three, four, five or all six CDRs from the non-human species and a framework region from a human immunoglobulin molecule.

A humanized antibody is a genetically engineered antibody in which the variable heavy and variable light CDRs from a non-human “donor” antibody are grafted into human “acceptor” antibody sequences (see for example, U.S. Pat. Nos. 5,530,101; 5,585,089; 5,225,539; 6,407,213; 5,859,205; and 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a non-human donor antibody and variable region framework sequences and constant regions, if present, entirely or substantially from human antibody sequences. Similarly, a humanized heavy chain has at least one, two, and usually all three CDRs entirely or substantially from a donor antibody heavy chain, and a heavy chain variable region framework sequence, and heavy chain constant region, if present, substantially from human heavy chain variable region framework and constant region sequences. Similarly, a humanized light chain has at least one, two, and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain.

As noted, humanized antibodies can be generated by replacing framework sequences of the variable region that are not directly involved in antigen binding with equivalent sequences from human variable regions. Those methods include isolating, manipulating, and expressing the nucleic acid sequences that encode all or part of variable regions from at least one of a heavy or light chain. Sources of such nucleic acid may be obtained from a hybridoma producing an antibody which binds to a target antigen and acts antagonistically against the target antigen. The recombinant DNA encoding the humanized antibody, or fragment thereof, can then be cloned into an appropriate expression vector. Each antibody light and heavy chain variable region consists of a framework region interrupted by the three corresponding CDRs. In one non-limiting embodiment, humanized antibodies are antibody molecules from non-human species having one, two, or all CDRs from the non-human species and a framework region from a human immunoglobulin molecule. Therefore, humanized antibodies can be generated by replacing framework sequences of the variable region that are not directly involved in antigen binding with equivalent sequences from human variable regions. Those methods include isolating, manipulating, and expressing the nucleic acid sequences that encode all or part of variable regions from at least one of a heavy or light chain. Sources of such nucleic acid are well known to those skilled in the art and, for example, may be obtained from a hybridoma producing an antibody against a target antigen, for example as described herein. The recombinant DNA encoding the humanized antibody, or fragment thereof, can then be cloned into an appropriate expression vector.

The humanized antibodies of the present disclosure can be produced by methods known in the art. For example, once an antibody is obtained, the variable regions can be sequenced, and the location of the CDRs and framework residues determined. See, for example, Kabat et al. (Sequences of Proteins of Immunological Interest, Fifth Edition, U.S. Department of Health and Human Services, NIH Publication No. 91-3242 (1991)); and Chothia et al. (J. Mol. Biol., 196:901-917 (1987)). The light and heavy chain variable regions can optionally be ligated to corresponding constant regions. CDR-grafted antibody molecules can be produced by CDR-grafting or CDR substitution. One, two, or all CDRs of an immunoglobulin chain can be replaced. For example, all of the CDRs of a particular antibody may be from at least a portion of a non-human animal (e.g., mouse, such as (but not limited to) CDRs shown herein), or only some of the CDRs may be replaced. It is only necessary to keep the CDRs which are required for specific and high binding affinity of the antibody to the target antigen. Once expressed, antibodies can be purified according to standard procedures of the art, including but not limited to HPLC purification, column chromatography, and gel electrophoresis. Methods for producing human antibodies include, but are not limited to, those shown in U.S. Pat. Nos. 4,634,664; 4,634,666; 5,877,397; 5,874,299; 5,814,318; 5,789,650; 5,770,429; 5,661,016; 5,633,425; 5,625,126; 5,569,825; 5,545,806; 5,877,218; 5,871,907; 5,858,657; 5,837,242; 5,733,743; and 5,565,332; and International Patent Application Publication Nos. WO 91/17271; WO 92/01047; and WO93/12227.

A fully human antibody can be obtained from a transgenic non-human animal (see, e.g., U.S. Pat. No. 5,633,425). Methods for producing fully human antibodies using either combinatorial approaches or transgenic animals transformed with human immunoglobulin loci are known in the art. Such fully human antibodies are expected to exhibit even fewer side effects than chimeric or humanized antibodies and to function in vivo as essentially endogenous human antibodies. In certain non-limiting embodiments, the claimed methods and procedures may utilize human antibodies produced by such techniques.

A CDR in a humanized or human antibody may be defined as “substantially derived from” or “substantially identical to” a corresponding CDR in a non-human antibody when at least 60%, or at least 65%, or at least 70%, or at least 75%, or at least 80%, or at least 85%, or at least 90%, or at least 95%, or 100% of corresponding residues (as defined by Kabat) are identical between the respective CDRs. In some non-limiting embodiments, a CDR in a humanized antibody or human antibody is substantially derived from or substantially identical to a corresponding CDR in a non-human antibody when there are no more than one, two, or three conservative amino acid substitutions in any given CDR. The variable region framework sequences of an antibody chain or the constant region of an antibody chain are “substantially from” a human variable region framework sequence or human constant region, respectively, when at least about 60%, at least about 65%, at least about 70%, at least about 75%, at least about 80%, at least about 85%, at least about 90%, at least about 95%, or about 100% of corresponding residues (as defined by Kabat numbering) are identical. As noted elsewhere herein, although humanized antibodies often incorporate all six CDRs (e.g., as defined elsewhere herein) from a non-human (e.g., mouse or rabbit) antibody, they can also be made with less than all of the non-human CDRs (e.g., at least 2, 3, 4, or 5).

The present disclosure provides, in certain non-limiting embodiments, antibodies to a target antigen in which the heavy chain variable region has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to a heavy chain variable sequence disclosed herein, and the light chain variable region has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to a light chain variable sequence disclosed herein. In some aspects, the antibody is a humanized antibody, and there is at least one murine backmutation in the heavy chain variable framework region. In other aspects, the antibody is a humanized antibody, and there is at least one murine backmutation in the light chain variable framework region.

Additionally, the disclosure provides antibodies in which the humanized heavy chain variable region comprises CDRs having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to the CDRs of a heavy chain variable sequence disclosed herein, and the humanized light chain variable region comprises CDRs having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to the CDRs of a light chain variable sequence disclosed herein.

Heavy and light chain variable regions of humanized antibodies can be linked to at least a portion of a human constant region, for example, for human antibody isotypes IgG1, IgG2, IgG3, or IgG4. Light chain constant regions can be lambda or kappa. Antibodies can be expressed as, for example (but not by way of limitation): tetramers containing two light and two heavy chains, as separate heavy chains, light chains, as Fab, Fab′, F(ab′)₂, and Fv, or as single chain antibodies in which heavy and light chain variable domains are linked through a spacer. All antibody isotypes are encompassed by the present disclosure, including (but not limited to) IgG (e.g., IgG1, IgG2, IgG3, IgG4), IgM, IgA (IgA1, IgA2), IgD, or IgE. The antibodies or antigen-binding portions thereof may be mammalian (e.g., mouse, rabbit, human) antibodies or antigen-binding portions thereof.

Humanized or chimeric antibodies are typically produced by recombinant expression. Recombinant polynucleotide constructs typically include an expression control sequence operably linked to the coding sequences of antibody chains, including naturally-associated or heterologous promoter regions. The expression control sequences may be eukaryotic promoter systems in vectors capable of transforming or transfecting eukaryotic host cells. Once the vector has been incorporated into the appropriate host, the host is maintained under conditions suitable for high level expression of the nucleotide sequences, and for the collection and purification of the cross-reacting antibodies.

Mammalian cells may be used as hosts for expressing nucleotide segments encoding immunoglobulins or fragments thereof. A number of suitable host cell lines capable of secreting intact heterologous proteins have been developed in the art, and include (but are not limited to) CHO cell lines (e.g., DG44), various COS cell lines, HeLa cells, HEK293 cells, L cells, and non-antibody-producing myelomas including Sp2/0 and NS0. In particular (but non-limiting) embodiments, the cells are non-human. Expression vectors for these cells can include expression control sequences, such as (but not limited to) an origin of replication, a promoter, an enhancer, and necessary processing information sites, such as (but not limited to) ribosome binding sites, RNA splice sites, polyadenylation sites, and transcriptional terminator sequences. Examples of expression control sequences include, but are not limited to, promoters derived from endogenous genes, cytomegalovirus, SV40, adenovirus, or bovine papillomavirus.

The present disclosure also encompasses nucleic acids encoding the present antibody or antigen-binding portion thereof that specifically binds to a target antigen as disclosed herein. The nucleic acid may be expressed in a cell to produce the presently disclosed antibody or antigen-binding portion thereof. The isolated nucleic acid of the present disclosure comprises, for example (but not by way of limitation), a sequence encoding a peptide that is at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% homologous to the monoclonal antibodies (mAbs) described elsewhere herein.

A nucleic acid encoding the present antibody or antigen-binding portion thereof may be introduced into an expression vector that can be expressed in a suitable expression system, followed by isolation or purification of the expressed antibody or antigen-binding portion thereof. Optionally, a nucleic acid encoding the present antibody or antigen-binding portion thereof can be translated in a cell-free translation system, e.g., see U.S. Pat. No. 4,816,567.

Antibodies or antigen binding portions against the presently-disclosed target antigens thereof can be produced by host cells transformed with DNA encoding light and heavy chains (or CDR portions thereof) of a desired antibody. Antibodies can be isolated and purified from these culture supernatants and/or cells using standard techniques. For example, a host cell may be transformed with DNA encoding the light chain, the heavy chain, or both, of an antibody. Recombinant DNA technology may also be used to remove some or all of the DNA encoding either or both of the light and heavy chains that is not necessary for binding, e.g., the constant region.

The present nucleic acids can be expressed in various suitable cells, including prokaryotic and eukaryotic cells, e.g., bacterial cells, (e.g., E. coli), yeast cells, plant cells, insect cells, and mammalian cells. A number of mammalian cell lines are known in the art and include (but are not limited to) immortalized cell lines available from the ATCC. Non-limiting examples of the cells include all cell lines of mammalian origin or mammalian-like characteristics, including but not limited to, parental cells, derivatives and/or engineered variants of monkey kidney cells (COS, e.g., COS-1, COS-7), HEK293, baby hamster kidney (BHK, e.g., BHK21), CHO, NS0, PerC6, BSC-1, human hepatocellular carcinoma cells (e.g., Hep G2), SP2/0, HeLa, Madin-Darby bovine kidney (MDBK) cells, myeloma cells, and lymphoma cells. The engineered variants include (for example but not by way of limitation) glycan profile modified and/or site-specific integration site derivatives.

The present disclosure also provides for cells comprising the nucleic acids described herein. The cells may be a hybridoma or transfectant. Examples of the cell types are discussed above.

Various techniques, such as production of chimeric or humanized antibodies, may involve procedures of antibody cloning and construction. The antigen-binding V_{L} (variable light chain) and V_{H} (variable heavy chain) sequences for an antibody of interest may be obtained by a variety of molecular cloning procedures, such as (but not limited to) RT-PCR, 5′-RACE, and cDNA library screening. The V_{L} and VW genes of an antibody from a cell that expresses a murine antibody can be cloned by PCR amplification and sequenced. To confirm their authenticity, the cloned V_{L} and V_{H} genes can be expressed in cell culture as a chimeric antibody, for example (but not by way of limitation) as described by Orlandi et al. (Proc. Natl. Acad. Sci. USA, 86: 3833 (1989)). Based on the V_{L} and VW gene sequences, a humanized antibody can then be designed and constructed as described by (for example but not by way of limitation) Leung et al. (Mol. Immunol., 32: 1413 (1995)).

The present disclosure further provides nucleic acids encoding any of the human or humanized heavy and light chains described herein. Typically, the nucleic acids also encode a signal peptide fused to the mature heavy and light chains. Coding sequences on nucleic acids can be in operable linkage with regulatory sequences to ensure expression of the coding sequences, such as (but not limited to) a promoter, enhancer, ribosome binding site, transcription termination signal, and the like. The present disclosure in further embodiments includes vectors which comprise the nucleic acids encoding heavy and light chains, and hosts cells which have been transfected with such vectors. The nucleic acids can be synthesized by, for example (but not by way of limitation), solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector. In certain non-limiting embodiments, the present antibody or antigen-binding portion thereof can be synthesized by solid phase procedures well known in the art.

Antibody fragments which recognize specific epitopes can be generated by known techniques. The antibody fragments are antigen binding portions of an antibody, such as (but not limited to) F(ab)₂, Fab′, Fab, Fv, scFv, and other fragments described herein or otherwise contemplated in the art. Other antibody fragments include, but are not limited to, the F(ab′)₂ fragments which can be produced by pepsin digestion of the antibody molecule and the Fab′ fragments, which can be generated by reducing disulfide bridges of the F(ab′)2 fragments. Alternatively, Fab′ expression libraries can be constructed to allow rapid and easy identification of monoclonal Fab′ fragments with the desired specificity. In certain non-limiting embodiments, the antibody fragment may be a fragment that is not an scFv fragment.

An scFv molecule comprises a V_{L} domain and a V_{H} domain. The V_{L} and V_{H} domains associate to form a target binding site. These two domains are further covalently linked by a peptide linker (L). Methods for making scFv molecules and designing suitable peptide linkers are known in the art and include (but are not limited to) those disclosed in U.S. Pat. Nos. 4,704,692 and 4,946,778, for example. An antibody fragment can be prepared by known methods, for example (but not by way of limitation), those disclosed by U.S. Pat. Nos. 4,036,945 and 4,331,647. Another form of an antibody fragment is a single-domain antibody (dAb), sometimes referred to as a single chain antibody. Techniques for producing single-domain antibodies are well known in the art

In certain non-limiting embodiments, the sequences of antibodies, such as (but not limited to) the Fc portions of antibodies, may be varied to optimize the physiological characteristics of the conjugates, such as (but not limited to) the half-life in serum. Methods of substituting amino acid sequences in proteins are widely known in the art, such as (but not limited to) by site-directed mutagenesis (e.g., Sambrook et al., Molecular Cloning, A laboratory manual, 2^{nd} Ed, 1989). In certain non-limiting embodiments, the variation may involve the addition or removal of one or more glycosylation sites in the Fc sequence (see, for example but not by way of limitation, U.S. Pat. No. 6,254,868).

The presently disclosed antibodies or antigen-binding fragments thereof may have a specific binding K_{D} to a target antigen of less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. Specific binding is detectably higher in magnitude and distinguishable from non-specific binding occurring to at least one unrelated target. For example (but not by way of limitation), the presently disclosed antibodies or antigen-binding fragments thereof have specific binding K_{D} to an epitope of a target antigen of less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹²M.

As mentioned above, the presently disclosed mAbs and mAb-derived compounds (e.g., antigen binding fragments) can be derivatized or linked to, e.g., conjugated to, therapeutic agents and/or diagnostic agents to form ADCs. For example, an antibody can be functionally linked, directly or indirectly, by covalent bonding or by noncovalent interactions to one or more other molecular entities, such as (but not limited to) another antibody, antibody fragment, a detectable agent, a cytotoxic agent, a pharmaceutical agent, a protein or peptide that can mediate association with another molecule (such as a streptavidin core region or a polyhistidine tag), amino acid linkers, spacers, bridges, signal sequences, immunogenic carriers, or ligands useful in protein purification, such as (but not limited to) glutathione-S-transferase, histidine tag, and staphylococcal protein A. Useful detectable agents with which a protein can be derivatized (or labeled) include (but are not limited to) fluorescent compounds, various enzymes, prosthetic groups, luminescent materials, bioluminescent materials, and radioactive materials. Non-limiting, exemplary fluorescent detectable agents include fluorescein, fluorescein isothiocyanate, rhodamine, and phycoerythrin. A protein or antibody can also be derivatized with detectable enzymes, such as (but not limited to) alkaline phosphatase, horseradish peroxidase, beta-galactosidase, acetylcholinesterase, glucose oxidase, and the like. A protein can also be derivatized with a prosthetic group (such as, but not limited to, streptavidin/biotin and avidin/biotin).

As noted, in certain non-limiting embodiments, the antibodies or fragments thereof may be used in combination with one or more therapeutic and/or diagnostic agents. Where the agent is attached to an antibody or fragment thereof to form an “antibody-drug conjugate” (“ADC”) to be administered by a method described herein, then the use of only non-cytotoxic, or minimally cytotoxic agents are contemplated. Non-cytotoxic agents may include, without limitation, immunomodulators, cytokines (and their inhibitors), chemokines (and their inhibitors), tyrosine kinase inhibitors, growth factors, hormones, and certain enzymes (i.e., those that do not induce local necrosis), or their inhibitors. Where the agentis co-administered either before, simultaneously with, or after the subcutaneous, intramuscular, or transdermal antibody formulation, then cytotoxic agents may be utilized. An agent may be administered as an immunoconjugate with a second antibody or fragment thereof, or may be administered as a free agent. The following discussion applies to both cytotoxic and non-cytotoxic agents.

Examples of therapeutic agents that can be conjugated to the antibody or antibody fragment (or is delivered separately) to form an ADC include, but are not limited to: 5-fluorouracil, aplidin, azaribine, anastrozole, anthracyclines, bendamustine, bleomycin, bortezomib, bryostatin-1, busulfan, calicheamycin, camptothecin, carboplatin, 10-hydroxycamptothecin, carmustine, celecoxib, chlorambucil, cisplatinum, Cox-2 inhibitors, CPT-11 SN-38, carboplatin, cladribine, camptothecans, cyclophosphamide, cytarabine, dacarbazine, docetaxel, dactinomycin, daunorubicin, doxorubicin, 2-pyrrolinodoxorubicine (2P-DOX), pro-2P-DOX, cyano-morpholino doxorubicin, doxorubicin glucuronide, epirubicin glucuronide, estramustine, epipodophyllotoxin, estrogen receptor binding agents, etoposide (VP16), etoposide glucuronide, etoposide phosphate, floxuridine (FUdR), 3′,5′-O-dioleoyl-FudR (FUdR-dO), fludarabine, flutamide, farnesyl-protein transferase inhibitors, gemcitabine, hydroxyurea, idarubicin, ifosfamide, L-asparaginase, lenolidamide, leucovorin, lomustine, mechlorethamine, melphalan, mercaptopurine, 6-mercaptopurine, methotrexate, mitoxantrone, mithramycin, mitomycin, mitotane, navelbine, nitrosourea, plicomycin, procarbazine, paclitaxel, pentostatin, PSI-341, raloxifene, semustine, streptozocin, tamoxifen, paclitaxel, temazolomide, transplatinum, thalidomide, thioguanine, thiotepa, teniposide, topotecan, uracil mustard, vinorelbine, vinblastine, vincristine, a vinca alkaloid, a tyrophostin, canertinib, dasatinib, erlotinib, gefitinib, imatinib, lapatinib, leflunomide, nilotinib, pazopanib, semaxinib, sorafenib, sunitinib, sutent, vatalanib, PCI-32765 (ibrutinib), PCI-45292, GDC-0834, LFM-A13, and RN486.

Examples of toxins include, but are not limited to, ricin, abrin, alpha toxin, saporin, ribonuclease (RNase; e.g., onconase), DNase I, Staphylococcal enterotoxin-A, pokeweed antiviral protein, gelonin, diphtheria toxin, Pseudomonas exotoxin, and Pseudomonas endotoxin.

Immunomodulators include, but are not limited to: cytokines, stem cell growth factors, lymphotoxins, hematopoietic factors, colony stimulating factors (CSF), interferons (IFN), erythropoietins, thrombopoietins, and combinations thereof. Specifically useful are lymphotoxins such as (but not limited to) tumor necrosis factor (TNF); hematopoietic factors such as (but not limited to) interleukin (IL); colony stimulating factors such as (but not limited to) granulocyte-colony stimulating factor (G-CSF) or granulocyte macrophage-colony stimulating factor (GM-CSF); interferons such as (but not limited to) interferons-alpha, -beta, -lambda, or -gamma; and stem cell growth factors such as (but not limited to) that designated “S1 factor.” Included among the cytokines are growth hormones such as (but not limited to): human growth hormone, N-methionyl human growth hormone, and bovine growth hormone; parathyroid hormone; thyroxine; insulin; proinsulin; relaxin; prorelaxin; glycoprotein hormones such as (but not limited to) follicle stimulating hormone (FSH), thyroid stimulating hormone (TSH), and luteinizing hormone (LH); hepatic growth factor; prostaglandin; fibroblast growth factor; prolactin; placental lactogen; OB protein; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as (but not limited to) NGF-beta; platelet-growth factor; transforming growth factors (TGFs) such as (but not limited to) TGF-alpha and TGF-beta; insulin-like growth factor-I and -II; erythropoietin (EPO); osteoinductive factors; interferons such as (but not limited to) interferon-alpha, -beta, -lambda, and -gamma; colony stimulating factors (CSFs) such as (but not limited to) macrophage-CSF (M-CSF); interleukins (ILs) such as (but not limited to) IL-1, IL-1alpha, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-23, IL-25; leukemia inhibitory factor (LIF); kit-ligand or FLT-3 ligand; angiostatin; thrombospondin; endostatin; tumor necrosis factor; and lymphotoxin. Chemokines which may be used include, but are not limited to): RANTES, MCAF, MIP1-alpha, MIP1-Beta, and IP-10.

In certain non-limiting embodiments, therapeutic radionuclides have a decay-energy in the range of 20 to 6,000 keV, such as (but not limited to) in the ranges of: 60 to 200 keV for an Auger emitter; 100-2,500 keV for a beta emitter; and 4,000-6,000 keV for an alpha emitter. Maximum decay energies of useful beta-particle-emitting nuclides may be, but are not limited to, 20-5,000 keV, 100-4,000 keV, or 500-2,500 keV Also included are radionuclides that substantially decay with Auger-emitting particles, such as (but not limited to): Co-58, Ga-67, Br-80m, Tc-99m, Rh-103m, Pt-109, In-111, Sb-119, 1-125, Ho-161, Os-189m, and Ir-192. Decay energies of useful beta-particle-emitting nuclides may be (for example but not by way of limitation): <1,000 keV, <100 keV, or <70 keV Also included are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227, and Fm-255. Decay energies of useful alpha-particle-emitting radionuclides include (but are not limited to): 2,000-10,000 keV; 3,000-8,000 keV; or 4,000-7,000 keV.

A connection between the antibody (or fragment thereof) and the linker can be via a number of different routes, such as (but not limited to): through a thioether bond, through a disulfide bond, through an amide bond, or through an ester bond. In one non-limiting embodiment, the connection between the antibody and the linker is formed between a thiol group of a cysteine residue of the antibody and a maleimide group of the linker. In some non-limiting embodiments, the interchain bonds of the antibody are converted to free thiol groups prior to reaction with the functional group of the linker. In some non-limiting embodiments, a cysteine residue is introduced into the heavy or light chain of an antibody and reacted with the linker. Positions for cysteine insertion by substitution in antibody heavy or light chains include (but are not limited to) those described in U.S. Patent Application Publication No. 2007/0092940 and International Patent Application Publication No. WO 2008/070593.

In some non-limiting embodiments, the ADCs of the present disclosure have the formula: Mab-(LU-D)ₙ, wherein Mab is a presently disclosed anti-target antigen antibody, or fragment thereof, LU is a Linker unit, and D is a Drug unit (i.e., the therapeutic or diagnostic agent). The subscript n ranges for example (but not by way of limitation) from 1 to 20 (i.e., 1, 2, 3, 4, 5, 6, 7, 8, 9, 10, 11, 12, 13, 14, 15, 16, 17, 18, 19, or 20), or more. Such conjugates comprise an anti-target antigen antibody covalently linked to at least one drug via a linker. The LU is connected at one end to the antibody and at the other end to a drug molecule. The skilled artisan will appreciate that in some aspects, the subscript n represents the number of drug-linkers on a singular antibody. In other aspects, n represents the average number of drug-linker molecules per antibody, e.g., the average number of drug-linkers per antibody in a reaction mixture or composition (e.g., pharmaceutical composition), and can be an integer or non-integer value. Accordingly, in some aspects, for compositions (e.g., pharmaceutical compositions), n represents the average drug loading of the antibody-drug conjugates in the composition, and n ranges from 1 to 20. In some non-limiting embodiments, the present disclosure provides antibody-linker conjugates have the formula: Mab-(LU)ₙ, wherein Mab is an anti-target antigen antibody, or fragment thereof, and LU is a Linker unit for linking a drug to the antibody. The subscript n ranges for example from 1 to 20 (i.e., 1, 2, 3, 4, 5, 6, 7, 8, 9, 10, 11, 12, 13, 14, 15, 16, 17, 18, 19, or 20), or more. Such conjugates comprise an anti-target antigen antibody covalently linked to one or more linkers, wherein the LU is connected at one end to the antibody and has a free end for connecting to a drug molecule. In some non-limiting embodiments, n is from about 1 to about 18 drugs per antibody. In some non-limiting embodiments, n is 1. In some non-limiting embodiments, n is 2. In some non-limiting embodiments, n is from about 2 to about 12 drugs per antibody. In some non-limiting embodiments, n is in a range of from about 2 to about 10, a range of from about 2 to about 8, a range of from about 2 to about 6, a range of from about 2 to about 5, a range of from about 2 to about 4, or a range of from about 2 to about 3 per antibody.

In certain non-limiting embodiments, a therapeutic and/or diagnostic agent may be covalently attached to an antibody or fragment thereof to form an immunoconjugate. In some non-limiting embodiments, the therapeutic and/or diagnostic agent may be attached to an antibody or fragment thereof via a carrier moiety. Carrier moieties may be attached, for example (but not by way of limitation) to reduced SH groups and/or to carbohydrate side chains. A carrier moiety can be attached at the hinge region of a reduced antibody component via disulfide bond formation. Alternatively, such agents can be attached using a heterobifunctional cross-linker, such as (but not limited to) N-succinyl 3-(2-pyridyldithio)propionate (e.g., see Yu et al., Int. J. Cancer, 56: 244 (1994). General techniques for such conjugation are well-known in the art; see, for example, Wong, Chemistry Of Protein Conjugation And Cross-Linking (CRC Press, 1991); Upeslacis et al., “Modification of Antibodies by Chemical Methods,” in Monoclonal Antibodies: Principles And Applications, Birch et al. (eds.), pages 187-230 (Wiley-Liss, Inc., 1995); and Price, “Production and Characterization of Synthetic Peptide-Derived Antibodies,” in Monoclonal Antibodies: Production, Engineering And Clinical Applicaion, Ritter et al. (eds.), pages 60-84 (Cambridge University Press, 1995). Alternatively, the carrier moiety can be conjugated via a carbohydrate moiety in the Fc region of the antibody.

Methods for conjugating functional groups to antibodies via an antibody carbohydrate moiety are well-known to those of skill in the art; see, for example, Shih et al., Int. J. Cancer, 41: 832 (1988); Shih et al., Int. J. Cancer, 46: 1101 (1990); and U.S. Pat. No. 5,057,313. The general method involves reacting an antibody having an oxidized carbohydrate portion with a carrier polymer that has at least one free amine function. This reaction results in an initial Schiff base (imine) linkage, which can be stabilized by reduction to a secondary amine to form the final conjugate.

Other methods of chemical conjugation of such moieties to biomolecules are well known in the art, and any such known method may be utilized to form an antibody conjugate that functions in accordance with the present disclosure. Such methods of immunoconjugate formation are disclosed, for example (but not by way of limitation), in U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240.

The combinations described herein can be administered to a cancer patient at any time following diagnosis. For example, the cancer patient can be treatment naive (e.g., has not received a cancer therapy for the diagnosed cancer). The cancer patient can be treatment naive for one cancer but can be diagnosed with one or more other cancers resulting from, for example, metastasis or malignancy. The cancer patient can be immune checkpoint naive for one or more cancers. The cancer patient can have a cancer that is refractory. In certain instances, the combinations described herein are administered as a first line therapy (e.g., the first therapy administered to a treatment naive cancer patient) to a patient in need thereof

It should also be appreciated that the combinations described herein for treating cancer can be co-administered with active agents other than those present in the combinations described herein (e.g., anti-cancer agents). Regimens for administration of a combination described herein, including the exemplary regimens set forth herein, can be modified as necessary to include administration of such active agents. Exemplary anti-cancer agents include but are not limited to those shown in US Published Patent Application 2020/0383961. Other examples of chemotherapeutic agent include but are not limited to hormones, hormonal analogues and antihormonals (e.g. tamoxifen, toremifene, raloxifene, fulvestrant, megestrol acetate, flutamide, nilutamide, bicalutamide, cyproterone acetate, finasteride, buserelin acetate, fludrocortisone, fluoxymesterone, medroxyprogesterone, octreotide, arzoxifene, pasireotide, vapreotide), aromatase inhibitors (e.g. anastrozole, letrozole, liarozole, exemestane, atamestane, formestane), LHRH agonists and antagonists (e.g. goserelin acetate, leuprolide, abarelix, cetrorelix, deslorelin, histrelin, triptorelin), antimetabolites (e.g. antifolates like methotrexate, pemetrexed, pyrimidine analogues like 5 fluorouracil, capecitabine, decitabine, nelarabine, and gemcitabine, purine and adenosine analogues such as mercaptopurine thioguanine, cladribine and pentostatin, cytarabine, fludarabine); antitumor antibiotics (e.g. anthracyclines like doxorubicin, daunorubicin, epirubicin and idarubicin, mitomycin-C, bleomycin dactinomycin, plicamycin, mitoxantrone, pixantrone, streptozocin); platinum derivatives (e.g. cisplatin, oxaliplatin, carboplatin, lobaplatin, satraplatin); alkylating agents (e.g. estramustine, meclorethamine, melphalan, chlorambucil, busulphan, dacarbazine, cyclophosphamide, ifosfamide, hydroxyurea, temozolomide, nitrosoureas such as carmustine and lomustine, thiotepa); antimitotic agents (e.g. vinca alkaloids like vinblastine, vindesine, vinorelbine, vinflunine and vincristine; and taxanes like paclitaxel, docetaxel and their formulations, larotaxel; simotaxel, and epothilones like ixabepilone, patupilone, ZK-EPO); topoisomerase inhibitors (e.g. epipodophyllotoxins like etoposide and etopophos, teniposide, amsacrine, topotecan, irinotecan) and miscellaneous chemotherapeutics such as amifostine, anagrelide, interferone alpha, procarbazine, mitotane, and porfimer, bexarotene, and celecoxib. The therapy may be a “platinum doublet” therapy, i.e., therapy with (i) a platinum compound such as cisplatin or carboplatin, plus (ii) a third-generation chemotherapy agent such as docetaxel, paclitaxel, vinorelbine, or gemcitabine.

In this context, “combination” or “combined” as used herein includes, without being limited to, a product that results from the mixing or combining of more than one active agent and includes both fixed and non-fixed (e.g., free) combinations (including kits) and uses, such as e.g., the simultaneous, concurrent, sequential, successive, alternate or separate use of the components or agents. The term “fixed combination” means that the active agents are both administered to a patient simultaneously in the form of a single entity or dosage. The term “non-fixed combination” means that the active agents are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active agents.

For example, simultaneous administration may include administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g., according to the agents used and the condition of the subject.

Accordingly, in one embodiment, in the method according to the present disclosure, the CGRP inhibitor as described herein is administered simultaneously, concurrently, sequentially, successively, alternately or separately with the PD-1 antagonist, PD-L1 antagonist, and/or CTLA-4 inhibitor as described herein.

Examples of routes of administration for the therapeutic compounds, administered separately or simultaneously, include, but are not limited to, oral, enterical, parenteral (e.g. intramuscular, intraperitoneal, intravenous, transdermal or subcutaneous injection, or implant), nasal, vaginal, rectal, or topical administration. The compounds of the present invention may be formulated, alone or together, in suitable dosage unit formulations containing conventional non-toxic pharmaceutically acceptable carriers, excipients and/or vehicles appropriate for each route of administration. Formulations may include solid, semi-solid or liquid dosage forms, such as lyophilisation, liquid solutions (e.g. injectable and infusible solutions), dispersions or suspensions, liposomes and suppositories. Certain embodiments include liquid formulations and lyophilisation. In the case of a lyophilisation, the lyophilisate may be reconstituted in a liquid, preferably water.

The compounds as described herein may be administered, for example, daily, 5 times a week, 3 times a week, 2 times a week, once a week, once in 2 weeks, once in 3 weeks, once in 4 weeks. The compounds may be administered once a week by i.v. infusion.

An administration regimen may include long-term treatment. By “long-term” is meant at least two weeks and preferably, several weeks, months or years of duration. Necessary modifications in this dosage regimen may be determined by one of ordinary skill in the art using only routine experimentation given the teachings herein (see Remington: The Science and Practice of Pharmacy, 23^{rd} Ed., Academic Press). The dosage can also be adjusted by the individual physician in the event of any complication. Administration may be daily, every second day, every third day, every fourth day, one day per week, two days per week, one day per two weeks, one day per three weeks, etc.

The compounds as described herein may be administered at therapeutically effective amounts in single or divided doses administered at appropriate time intervals. A therapeutically effective amount refers to an amount effective at dosages and for periods of time necessary to achieve the desired therapeutic result and is the minimum amount necessary to prevent, ameliorate, or treat a disease or disorder. A therapeutically effective amount of the compounds according to the present invention may vary according to factors such as the disease state, age, sex, and weight of the individual, and the ability of the compound to elicit a desired response in the individual. A therapeutically effective amount is also one in which any toxic or detrimental effects of the compound is outweighed by the therapeutically beneficial effects. A therapeutically effective dose preferably inhibits a measurable parameter, e.g., a tumor growth rate by at least about 20%, more preferably by at least about 40%, even more preferably by at least about 60%, and still more preferably by at least about 80% relative to untreated subjects or relative to a preceding untreated period of the same subject that is to be treated.

The active agents may be administered in such doses which are therapeutically effective in monotherapy, or in such doses which are lower or higher than the doses used in monotherapy, but when combined result in a desired (jointly) therapeutically effective amount. The amount of the bispecific binding molecules of the invention required for use in treatment may be adapted to the particular binding molecule selected, the route of administration, the nature of the condition being treated and the age and condition of the patient and will be ultimately at the discretion of the attendant physician or clinician. Also, the dosage of the binding molecules of the invention may be adapted depending on the target cell, tumor, tissue, graft, or organ.

The present antibodies or antigen-binding portions thereof can be formulated into compositions for delivery to a mammalian subject. The composition can be administered alone and/or mixed with a pharmaceutically acceptable vehicle or excipient. Suitable vehicles are, for example (but not by way of limitation), water, saline, dextrose, glycerol, ethanol, or the like, and combinations thereof. In addition, the vehicle can contain minor amounts of auxiliary substances such as (but not limited to) wetting or emulsifying agents, pH buffering agents, or adjuvants. The compositions of the present disclosure can also include ancillary substances, such as (but not limited to) pharmacological agents, cytokines, or other biological response modifiers.

Furthermore, the compositions can be formulated into compositions in either neutral or salt forms. Pharmaceutically acceptable salts include (but are not limited to) the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, and procaine.

Compositions can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, degree of immunoprotection desired, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The compositions can be combined with a pharmaceutically acceptable carrier (excipient) to form a pharmacological composition. Pharmaceutically acceptable carriers can contain a physiologically acceptable compound that acts to, for example but not by way of limitation) stabilize or increase or decrease the absorption or clearance rates of the pharmaceutical compositions. Physiologically acceptable compounds can include, for example but not by way of limitation: carbohydrates, such as glucose, sucrose, or dextrans; antioxidants, such as ascorbic acid or glutathione; chelating agents; low molecular weight proteins; detergents; liposomal carriers; excipients; or other stabilizers and/or buffers. Other physiologically acceptable compounds include (but are not limited to) wetting agents, emulsifying agents, dispersing agents, or preservatives.

When administered orally, the present compositions may be protected from digestion. This can be accomplished either by complexing the antibody or antigen-binding portion thereof with a composition to render it resistant to acidic and enzymatic hydrolysis or by packaging the antibody or antigen-binding portion thereof in an appropriately resistant carrier such as (but not limited to) a liposome, e.g., such as shown in U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The present compositions can also be administered in sustained delivery or sustained release mechanisms. For example, biodegradeable microspheres or capsules or other biodegradeable polymer configurations capable of sustained delivery of a peptide can be included herein.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include (for example but not by way of limitation) air jet nebulizers.

Antibodies or mAb-derived compounds can be delivered alone or as pharmaceutical compositions by any means known in the art, such as (but not limited to) systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

In one aspect, the pharmaceutical formulations comprising compositions or nucleic acids, antibodies or fragments thereof are incorporated in lipid monolayers or bilayers, such as (but not limited to) liposomes, such as shown in U.S. Pat. Nos. 6,110,490; 6,096,716; 5,283,185; and 5,279,833. In other aspects, non-limiting embodiments of the disclosure include formulations in which the polypeptides or nucleic acids have been attached to the surface of the monolayer or bilayer of the liposomes. Liposomes and liposomal formulations can be prepared according to standard methods and are also well known in the art, such as (but not limited to) those disclosed in U.S. Pat. Nos. 4,235,871; 4,501,728; and 4,837,028.

In one aspect, the compositions are prepared with carriers that will protect the antibody or fragment thereof against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The subject antibodies and fragments thereof in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active compound is combined in a mixture with one or more pharmaceutically suitable excipients. Sterile phosphate-buffered saline is one non-limiting example of a pharmaceutically suitable excipient.

Non-limiting examples of routes of administration of the compositions described herein include parenteral injection, e.g., by subcutaneous, intramuscular, or transdermal delivery. Other forms of parenteral administration include (but are not limited to) intravenous, intraarterial, intralymphatic, intrathecal, intraocular, intracerebral, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives.

Formulated compositions comprising the active agents can be used (for example but not by way of limitation) for subcutaneous, intramuscular, or transdermal administration. Compositions can be presented in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents. Alternatively, the compositions can be in powder form for constitution with a suitable vehicle, e.g., sterile pyrogen-free water, before use.

The compositions may be administered in solution. The formulation thereof may be in a solution having a suitable pharmaceutically acceptable buffer, such as (but not limited to) phosphate, Tris (hydroxymethyl) aminomethane-HCl, or citrate, and the like. Buffer concentrations should be in the range of 1 to 100 mM. The formulated solution may also contain a salt, such as (but not limited to) sodium chloride or potassium chloride in a concentration of 50 to 150 mM. An effective amount of a stabilizing agent such as (but not limited to) mannitol, trehalose, sorbitol, glycerol, albumin, a globulin, a detergent, a gelatin, a protamine, or a salt of protamine may also be included.

Exemplary, non-limiting ranges for a therapeutically or prophylactically effective amount of an antibody or mAb-derived compound of the present disclosure antibody-drug conjugate include a range of from about 0.001 mg/kg of the subject's body weight to about 100 mg/kg of the subject's body weight, such as but not limited to a range of from about 0.01 mg/kg to about 50 mg/kg, a range of from about 0.1 mg/kg to about 50 mg/kg, a range of from about 0.1 mg/kg to about 40 mg/kg, a range of from about 1 mg/kg to about 30 mg/kg, a range of from about 1 mg/kg to about 20 mg/kg, a range of from about 2 mg/kg to about 30 mg/kg, a range of from about 2 mg/kg to about 20 mg/kg, a range of from about 2 mg/kg to about 15 mg/kg, a range of from about 2 mg/kg to about 12 mg/kg, a range of from about 2 mg/kg to about 10 mg/kg, a range of from about 3 mg/kg to about 30 mg/kg, a range of from about 3 mg/kg to about 20 mg/kg, a range of from about 3 mg/kg to about 15 mg/kg, a range of from about 3 mg/kg to about 12 mg/kg, or a range of from about 3 mg/kg to about 10 mg/kg, or a range of from about 10 mg to about 1500 mg as a fixed dosage.

The composition is formulated to contain an effective amount of the presently disclosed active agent (i.e., mAbs, mAb-derived compounds, ADCs, or small molecules), wherein the amount depends on the animal to be treated and the condition to be treated. In certain non-limiting embodiments, the active agent is administered at a dose ranging from about 0.001 mg to about 10 g, from about 0.01 mg to about 10 g, from about 0.1 mg to about 10 g, from about 1 mg to about 10 g, from about 1 mg to about 9 g, from about 1 mg to about 8 g, from about 1 mg to about 7 g, from about 1 mg to about 6 g, from about 1 mg to about 5 g, from about 10 mg to about 10 g, from about 50 mg to about 5 g, from about 50 mg to about 5 g, from about 50 mg to about 2 g, from about 0.05 μg to about 1.5 mg, from about 10 μg to about 1 mg protein, from about 30 μg to about 500 μg, from about 40 μg to about 300 μg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, or from about 1 mg to about 2 mg. The specific dose level for any particular subject depends upon a variety of factors, including (but not limited to) the activity of the specific peptide, the age, body weight, general health, sex, diet, time of administration, route of administration, and rate of excretion, the drug combination, and the severity of the particular disease undergoing therapy.

The dosage of an administered active agent for humans will vary depending upon factors such as (but not limited to) the patient's age, weight, height, sex, general medical condition, and previous medical history. In certain non-limiting embodiments, the recipient is provided with a dosage of the active agent that is in the range of from about 1 mg to about 1000 mg as a single infusion or single or multiple injections, although a lower or higher dosage also may be administered. In certain non-limiting embodiments, the dosage may be in the range of from about 25 mg to about 100 mg of the active agent per square meter (m²) of body surface area for a typical adult, although a lower or higher dosage also may be administered. Non-limiting examples of dosages of the active agent that may be administered to a human subject further include 1 to 500 mg, 1 to 70 mg, or 1 to 20 mg, although higher or lower doses may be used. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In some non-limiting embodiments, the effective amount of an active agent sufficient to treat a particular cancer is in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 75 μM, about 80 μM, about 90 μM, about 100 μM, about 125 μM, about 150 μM, about 175 μM, about 200 μM, about 250 μM, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

The active agent can be present in an amount as a measure with regards to the weight of the patient in need thereof. For example, the active agent can be present in an amount of about: 0.1 mg/kg to about 50 mg/kg, 0.1 mg/kg to about 40 mg/kg, 0.1 mg/kg to about 30 mg/kg, 0.1 mg/kg to about 25 mg/kg, 0.1 mg/kg to about 20 mg/kg, 0.1 mg/kg to about 15 mg/kg, 0.1 mg/kg to about 10 mg/kg, 0.1 mg/kg to about 7.5 mg/kg, 0.1 mg/kg to about 5 mg/kg, 0.1 mg/kg to about 2.5 mg/kg, or about 0.1 mg/kg to about 1 mg/kg. The active agent can be present in an amount of about: 0.5 mg/kg to about 50 mg/kg, 0.5 mg/kg to about 40 mg/kg, 0.5 mg/kg to about 30 mg/kg, 0.5 mg/kg to about 25 mg/kg, 0.5 mg/kg to about 20 mg/kg, 0.5 mg/kg to about 15 mg/kg, 0.5 mg/kg to about 10 mg/kg, 0.5 mg/kg to about 7.5 mg/kg, 0.5 mg/kg to about 5 mg/kg, 0.5 mg/kg to about 2.5 mg/kg, or about 0.5 mg/kg to about 1 mg/kg. The active agent can be present in an amount of about 0.5 mg/kg to about 5 mg/kg or about 0.1 mg/kg to about 10 mg/kg. The active agent can be present in an amount of about 0.1 mg/kg to about 20 mg/kg or about 0.1 mg/kg to about 30 mg/kg.

In still other embodiments, the active agent can be present at an amount of about: 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, or 50 mg/kg. The active agent can be present at an amount of about: 1 mg/kg, 2 mg/kg, 3 mg/kg, 5 mg/kg, 10 mg/kg, 15 mg/kg, 20 mg/kg, 25 mg/kg, or 30 mg/kg. The active agent can be present at an amount of about: 3 mg/kg, 10 mg/kg, 20 mg/kg, or 30 mg/kg.

The active agent can be present in the combination at an amount of about: 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, or 200 mg. The active agent can be present in the combination at an amount of about: 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. The active agent can be present in the combination at an amount of about 1000 mg to about 2000 mg. The active agent can be present in the combination at an amount of about: 1 mg to about 10 mg, 10 mg to about 20 mg, 25 mg to about 50 mg, 30 mg to about 60 mg, 40 mg to about 50 mg, 50 mg to about 100 mg, 75 mg to about 150 mg, 100 mg to about 200 mg, 200 mg to about 500 mg, 500 mg to about 1000 mg, 1000 mg to about 1200 mg, 1000 mg to about 1500 mg, 1200 mg to about 1500 mg, or 1500 to about 2000 mg.

The active agent can be present in the combination in an amount of about 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 400 mg/mL, or 500 mg/mL. In one embodiment, the active agent is present in the combination in an amount of about: 1 mg/mL to about 10 mg/mL, 5 mg/mL to about 10 mg/mL, 5 mg/mL to about 15 mg/mL, 10 mg/mL to about 25 mg/mL; 20 mg/mL to about 30 mg/mL; 25 mg/mL to about 50 mg/mL, or 50 mg/mL to about 100 mg/mL.

In some non-limiting methods, the patient is administered the active agent every one, two, three, or four weeks, for example. The dosage depends on the frequency of administration, condition of the patient, response to prior treatment (if any), whether the treatment is prophylactic or therapeutic, and whether the disorder is acute or chronic, among other factors.

Administration can be (for example but not by way of limitation) parenteral, intravenous, oral, subcutaneous, intra-arterial, intracranial, intrathecal, intraperitoneal, topical, intranasal, or intramuscular. Administration can also be localized directly into a tumor. Administration into the systemic circulation by intravenous or subcutaneous administration is typical. Intravenous administration can be, for example (but not by way of limitation), by infusion over a period such as (but not limited to) 30-90 min or by a single bolus injection.

The frequency of administration depends on the half-life of the active agent in the circulation, the condition of the patient, and the route of administration, among other factors. The frequency can be daily, weekly, monthly, quarterly, or at irregular intervals in response to changes in the patient's condition or progression of the disorder treated. An exemplary (but non-limiting) frequency for intravenous administration is between twice a week and quarterly over a continuous course of treatment, although more or less frequent dosing is also possible. Other exemplary (but non-limiting) frequencies for intravenous administration are between once weekly or once monthly over a continuous course of treatment, although more or less frequent dosing is also possible. For subcutaneous administration, an exemplary (but non-limiting) dosing frequency is daily to monthly, although more or less frequent dosing is also possible. The number of dosages administered may depends on the severity and temporal nature of the cancer disorder

In certain non-limiting embodiments, pharmaceutical compositions for parenteral administration are sterile, substantially isotonic, and manufactured under GMP conditions. Pharmaceutical compositions can be provided in unit dosage form (i.e., the dosage for a single administration). Pharmaceutical compositions can be formulated using one or more physiologically acceptable carriers, diluents, excipients, or auxiliaries. The formulation depends on the route of administration chosen. For injection, the active agent can be formulated in aqueous solutions, such as (but not limited to) in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, the active agent can be in lyophilized form for constitution with a suitable vehicle, e.g., sterile pyrogen-free water, before use. The concentration of the active agent in a liquid formulation can be for example (but not by way of limitation) 0.01-10 mg/ml, such as 1.0 mg/ml.

Several non-limiting embodiments also encompass variants of the antibodies or antibody fragments used in the methods disclosed herein, comprising one or more amino acid residue substitutions in the V_{L} domain and/or V_{H} domain thereof. Several non-limiting embodiments also encompass variants of the above-described antibodies with one or more additional amino acid residue substitutions in one or more V_{L} CDRs and/or one or more V_{H} CDRs. The variants generated by introducing substitutions in the V_{H} domain, V_{H} CDRs, V_{L} domain, and/or V_{L} CDRs described herein can be tested in vitro and in vivo, for example, for its ability to bind to target antigens (by, e.g., immunoassays including, but not limited to, ELISAs and BIAcore).

The present disclosure also encompasses nucleic acids which encode the presently disclosed mAbs and mAb-derived compounds that specifically bind to the target antigens of the present disclosure. The nucleic acid may be expressed in a cell to produce the presently disclosed mAbs and mAb-derived compounds. For example, the isolated nucleic acid of the present disclosure comprises a sequence encoding a peptide that is at least about 70%, at least about 75%, at least about 80%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% identical to an amino acid sequence of a mAb or mAb-derived compound described herein. Nucleic acids encoding the present mAbs and mAb-derived compounds may be introduced into an expression vector that can be expressed in a suitable expression system, followed by isolation or purification of the expressed antibody or antigen-binding portion thereof. Optionally, nucleic acid encoding the mAb or mAb-derived compound can be translated in a cell-free translation system, such as (but not limited to) a system as disclosed in U.S. Pat. No. 4,816,567.

Antibodies or antibody fragments used in the present methods can be produced by host cells transformed with DNA encoding light and heavy chains (or CDR portions thereof) of a desired antibody. Antibodies can be isolated and purified from these culture supernatants and/or cells using standard techniques. For example, a host cell may be transformed with DNA encoding the light chain, the heavy chain, or both, of an antibody. Recombinant DNA technology may also be used to remove some or all of the DNA encoding either or both of the light and heavy chains that is not necessary for binding, e.g., the constant region.

Some non-limiting embodiments provided herein include kits. In some non-limiting embodiments, a kit can include any of the active agents as described or otherwise contemplated herein, such as (but not limited to) a humanized antibody or humanized binding fragment thereof. In some non-limiting embodiments, the antibody or binding fragment thereof is lyophilized. In some non-limiting embodiments, the antibody or binding fragment thereof is in aqueous solution, or other carrier as described herein. In some non-limiting embodiments, the kit includes a pharmaceutical carrier for administration of the antibody. In some non-limiting embodiments, the kit also includes a chemotherapeutic agent. Certain non-limiting embodiments of the present disclosure include kits containing components suitable for treatments or diagnosis. Exemplary kits may contain at least one CGRP-inhibiting antibody and at least one or more checkpoint inhibitors (e.g., PD-1- or PD-L1-inhibiting antibody, or binding fragments thereof or a CTLA-4-inhibiting antibody or fragment, or small molecule active agent), as described herein. A device capable of delivering the kit components by injection, for example, a syringe for subcutaneous injection, may be included in some non-limiting embodiments. Where transdermal administration is used, a delivery device such as hollow microneedle delivery device may be included in the kit in some non-limiting embodiments. Exemplary transdermal delivery devices are known in the art, such as (but not limited to) a hollow Microstructured Transdermal System (e.g., 3M Corp.), and any such known device may be used. The kit components may be packaged together or separated into two or more containers. In some non-limiting embodiments, the containers may be vials that contain sterile, lyophilized formulations of a composition that are suitable for reconstitution. A kit may also contain one or more buffers suitable for reconstitution and/or dilution of other reagents. Alternatively, the antibody or fragment may be delivered and stored as a liquid formulation. Other containers that may be used include, but are not limited to, a pouch, tray, box, tube, or the like. Kit components may be packaged and maintained sterilely within the containers. Another component that can be included is instructions for the use of the kit for treatment of certain diseases or conditions or for the diagnosis of such.

### Target Cancers

The cancer treated using the methods disclosed herein can be a solid tumor. The cancer can be a hematological cancer. In certain instances, the cancer is a solid tumor selected from the group consisting of squamous cell carcinoma, non-squamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST).

For example, in one embodiment the present disclosure is directed to a method of treating breast cancer by administering a therapeutically effective amount of a combination described herein, including for example a CGRP inhibitor and a PD-1- or PD-L1 antagonist, or a CGRP inhibitor and a CTLA-4 inhibitor. The breast cancer can be HER2 negative breast cancer. The breast cancer can be a HER2 positive breast cancer. The breast cancer can be triple-negative breast cancer. In some embodiments the breast cancer is a Stage IA or Stage D3 cancer. In another embodiment, the breast cancer is a Stage IIA or Stage IIB cancer. In still another embodiment, the breast cancer is a Stage IIIA, Stage IIIB, or Stage IIIC cancer. In yet another embodiment, the breast cancer is a Stage IV cancer.

In other embodiments, the cancer is a hematological cancer selected from lymphoma, Non-Hodgkin's lymphoma (NHL), Hodgkin's Lymphoma, Reed-Sternberg disease, multiple myeloma (MM), acute myelogenous leukemia (AML), chronic myelogenous leukemia (CIVIL), acute lymphocytic leukemia, (ALL), or chronic lymphocytic leukemia (CLL).

In certain embodiments, the oncological or hyperproliferative disease, in particular cancer or a tumor disease, treated with the combination therapy as disclosed herein, includes but is not limited to, a solid tumor, a hematological cancer (e.g., leukemia, lymphoma, myeloma, e.g., multiple myeloma), and a metastatic lesion. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma) and pancreas, as well as adenocarcinomas which include malignancies such as colon cancers, rectal cancer, renal-cell carcinoma, liver cancer, non-small cell lung cancer, cancer of the small intestine and cancer of the esophagus. The cancer may be at an early, intermediate, late stage or metastatic cancer.

As used herein, “hyperproliferative disease” refers to conditions wherein cell growth is increased over normal levels. For example, hyperproliferative diseases or disorders include malignant diseases (e.g., esophageal cancer, colon cancer, biliary cancer) and non-malignant diseases (e.g., atherosclerosis, benign hyperplasia, benign prostatic hypertrophy).

In one embodiment, the cancer is chosen from a lung cancer (e.g. NSCLC (e.g., a NSCLC with squamous and/or non-squamous histology, or a NSCLC adenocarcinoma)), a melanoma (e.g., an advanced melanoma), a renal cancer (e.g., a renal cell carcinoma), a liver cancer, a myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), a colorectal cancer, a pancreatic cancer, a head and neck cancer (e.g., head and neck squamous cell carcinoma (HNSCC), anal cancer, gastro-esophageal cancer, thyroid cancer, cervical cancer, a lymphoproliferative disease (e.g., a post-transplant lymphoproliferative disease) or a hematological cancer, T-cell lymphoma, B-cell lymphoma, a non-Hodgkin lymphoma, or a leukemia (e.g., a myeloid leukemia or a lymphoid leukemia).

In one embodiment, the cancer is a melanoma, e.g., an advanced melanoma. In one embodiment, the cancer is an advanced or unresectable melanoma that does not respond to other therapies. In other embodiments, the cancer is a melanoma with a BRAF mutation (e.g., a BRAF V600 mutation). In another embodiment, the cancer is a hepatocarcinoma, e.g., an advanced hepatocarcinoma, with or without a viral infection, e.g., a chronic viral hepatitis. In another embodiment, the cancer is a prostate cancer, e.g., an advanced prostate cancer. In another embodiment, the cancer is a myeloma, e.g., multiple myeloma. In another embodiment, the cancer is a renal cancer, e.g., a renal cell carcinoma (RCC) (e.g., a metastatic RCC or clear renal cell carcinoma CCRCC)).

### CGRP Inhibition

The treatment methods of the present disclosure require that the CGRP expression, activity, release, or receptor signaling is inhibited. As used herein, “inhibition of CGRP” refers to the inhibition of CGRP expression, CGRP activity, CGRP release, or CGRP receptor signaling. In certain embodiments of the present disclosure, the combination therapy may include an anti-CGRP active agent comprising a CGRP receptor-binding inhibiting antibody such as, but not limited to, erenumab (AMG 334), or binding portions thereof, or a CGRP-binding antibody such as, but not limited to, eptinezumab (ALD403), fremanezumab (TEV-48125), or galcanezumab (LY2951742), or binding portions thereof.

In certain embodiments, the active agent comprises a small molecule, antibody, or antibody fragment that inhibits CGRP. Examples of molecules (including small molecules) that inhibit CGRP and which can be used herein include, but are not limited to, atogepant, olcegepant, obrogepant, rimegepant (BMS-927711), avitriptan, eletriptan, naratriptan, rizatriptan, telcacepant, ubrogepant, sumatriptan, zolmitriptan, BIBN4096, BI44370, BMS-694153, and MK-3207.

In some embodiments, the CGRP inhibitor is selected from the compounds disclosed in, but not limited to, U.S. Pat. Nos. 11,111,289; 11,083,724; 11,066,386; 11,027,018; 10,899,826; 10,888,561; 10,668,132; 10,533,048; 10,370,425; 10,336,726; 10,300,056; 10,287,337; 10,272,077; 10,266,587; 10,259,812; 10,214,582; 10,208,112; 10,179,809; 10,166,226; 9,969,775; 9,951,115; 9,925,178; 9,862,771; 9,850,246; 9,833,448; 9,802,935; 9,745,373; 9,718,845; 9,708,297; 9,695,176; 9,585,940; 9,505,838; 9,499,545; 9,499,541; 9,409,916; 9,365,648; 9, 296,750; 9,227,973; 9,193,776; 9,102,731; 9,073,991; 8,993,588; 8,952,014; 8,895,752; 8,778,957; 8,765,763; 8,629,137; 8,586,045; 8,173,655; 8,163,752; 8,148,390; 8,143,266; and 8,044,043.

In certain embodiments, the active agent, such as an RNAi, inhibits the expression of CGRP, for example, by at least 10%, at least 20%, at least 30%, at least 40%, at least 50%, at least 60%, at least 70%, at least 80%, at least 90%, or more as compared to an appropriate control.

In certain embodiments, the active agent inhibits the function of CGRP, for example, by at least 10%, at least 20%, at least 30%, at least 40%, at least 50%, at least 60%, at least 70%, at least 80%, at least 90%, or more as compared to an appropriate control.

### PD-1 Antagonism

The term “PD-1 antagonist” refers to a substance (e.g., compound, nucleic acid, polypeptide, antibody) that decreases, inhibits, blocks, abrogates or interferes with the activity, binding of PD-1 with PD-1 ligand, or expression of PD-1 or PD-1 ligand, including variants, isoforms, species homologs of human PD-1 or PD-L1 (e.g., mouse) and analogs that have at least one common epitope with PD-1 or PD-L1. Such inhibitors include molecules and macromolecules such as, for example, small molecule compounds, nucleic acids, polypeptides, antibodies, peptibodies, diabodies, minibodies, single-chain variable fragments (ScFv), and fragments or variants thereof as described elsewhere herein. Thus, a PD-1 ligand inhibitor (e.g., and inhibitor of either PD-L1 or PD-L2) as used herein refers to any moiety that antagonizes PD-1 activity, its binding to PD-1 ligand, or its expression. A PD-L1 inhibitor as used herein refers to any moiety that antagonizes PD-L1 activity, its binding to PD-1, or its expression. PD-1 inhibitor efficacy can be measured, for example, by its inhibitor concentration at 50% (half-maximal inhibitor concentration or IC₅₀).

Examples of substances that inhibit or antagonize PD-1 activity (either by binding to PD-1 or to PD-1L) which may be used herein include but are not limited to the antibodies pembrolizumab, nivolumab, pidilizumab, toripalimab, balstilimab (AGEN2034), cemiplimab, dostarlimab, spartalizumab, camrelizumab, sintilimab, tislelizumab, AMP-224, AMP-514 (MEDI0680), BCD100, BMS-pep57, BMS-936559, INCMGA00012, PDR-001, REGN2810, SAR-439684, and SHR-1210. PD-1 inhibitors as disclosed in U.S. Pat. No. 10,577,422, which may be used herein include but are not limited to PD-1 mAb 1, PD-1 mAb 2, PD-1 mAb 3, PD-1 mAb 4, PD-1 mAb 5, PD-1 mAb 6, PD-1 mAb 7, PD-1 mAb 8, PD-1 mAb 9, PD-1 mAb 10, PD-1 mAb 11, PD-1 mAb 12, PD-1 mAb 13, PD-1 mAb 14, or PD-1 mAb 15, including humanized and chimeric variants thereof, and PD-1 binding fragments thereof. The PD-1 inhibitor can be a polypeptide (e.g., macrocyclic polypeptide) such as those exemplified in U.S. Patent Application Publication No.: 2014/0294898, which is incorporated herein by reference in its entirety and for all purposes. Examples of PD-L1 inhibitors include exemplary compounds and compositions described herein, including but not limited to the antibodies durvalumab, avelumab, atezolizumab, AUNP-12, CA-170 (AUPM-170; Curis, Inc.), STI-1110, STI-A1010, STI-A1011, STI-A1012, STI-A1013, STI-A1014, and STI-A1015.

In some embodiments, the PD-1 and/or PD-1L inhibitor which may be used herein is selected from the group consisting of AP-106, AP-105, BMS-8, BMS-57, BMS-71, BMS-99, BMS-103, BMS-142, BMS-200, BMS-202, BMS-242, BMS-1001, BMS-1166, MSB-2311, CBT-501, avelumab, AK-105, IO-102, IO-103, CX-072, SHR-1316, JTX-4014, GNS-1480, recombinant humanized anti-PD1 mAb (Shanghai Junshi Biosciences), BGB-A317, recombinant humanized anti-PD-1 mAb (Bio-Thera Solutions), Probody targeting PD-1 (CytomX), XmAb-20717, FS-118, PSI-001, SN-PDL01, SN-PD07, PD-1 modified TILs (Sangamo Therapeutics), PRS-332, FPT-155, TSR-042, REGN-1979, FAZ-053, PD-1/CTLA-4 bispecific antibody (MacroGenics), MGA-012, MGD-013, M-7824, PD-1 based bispecific antibody (Beijing Hanmi Pharmaceutical), AK-112, AK-106, AK-104, AK-103, BI-754091, ENUM-244C8, MCLA-145, MCLA-134, anti-PD1 oncolytic monoclonal antibody (Transgene SA), IBI-308, WBP-3155, JNJ-63723283, SSI-361, CBT-502, dual targeting anti-PD-1/LAG-3 mAbs (TESARO), dual targeting anti-PD-1/TIM-3 mAbs (TESARO), PF-06801591, LY-3300054, pembrolizumab biosimilar, nivolumab biosimilar, KY-1003, STI-1014, GLS-010, AM-0001, GX-P2, KD-033, PD-L1/BCMA bispecific antibody (Immune Pharmaceuticals), anti-PD-1NEGF-A DARPins (Molecular Partners), mDX-400, NP-12, ALN-PDL, siRNA loaded dendritic cell vaccine (Alnylam Pharmaceuticals), GB-226, PD-L1 targeting CAR-TNK-based immunotherapy (TNK Therapeutics/NantKwest), INSIX RA, INDUS-903, AMP-224, anti-CTLA-4/anti-PD-1 bispecific humanized antibody (Akeso Biopharma), and GX-D1.

In some embodiments, the PD-1 and/or PD-1L inhibitors which may be used herein is selected from the compounds disclosed in published PCT applications WO15033303, WO15036927, and WO15044900, published US patent applications US2020216498, US20180044350, US20180044329, US20180044305, US20180044304; US2015073024, US2016194295, US2016222060, and U.S. Pat. Nos. 8,907,053, 9,044,442, 9,096,642, 9,233,940, 9,308,236; 9,422,339; 9,732,119; 9,771,338; 9,783,578; 9,809,625; 9,850,225; 9,850,283; 9,861,680; 9,872,852; 9,879,046; 9,944,678; 10,160,736; 10,173,989; 10,538,555; 10,577,422, 10,590,093; 10,781,189, and 10,961,205, each of which is hereby incorporated herein by reference in its entirety.

### CTLA-4 Inhibition

The term “CTLA-4 inhibitor” refers to a moiety (e.g., compound, nucleic acid, polypeptide, antibody) that decreases, inhibits, blocks, abrogates or interferes with the activity or expression of Cytotoxic T-lymphocyte-Associated protein 4 (CTLA-4), including variants, isoforms, species homologs of human CTLA-4 (e.g., mouse) and analogs that have at least one common epitope with CTLA-4. A CTLA-4 inhibitor includes molecules and macromolecules such as, for example, compounds, nucleic acids, polypeptides, antibodies, peptibodies, diabodies, minibodies, single-chain variable fragments (ScFv), and fragments or variants thereof. Thus, a CTLA-4 inhibitor as used herein refers to any moiety that antagonizes CTLA-4 activity or expression. CTLA-4 inhibitor efficacy can be measured, for example, by its inhibitor concentration at 50% (half-maximal inhibitor concentration or IC₅₀). CTLA-4 inhibitors include exemplary compounds and compositions described herein. A CTLA-4 antibody refers to a CTLA-4 inhibitor which is a monoclonal or polyclonal antibody, or a CTLA-4-binding fragment thereof, as described herein. Non-limiting examples of CTLA-4 inhibitors which may be used herein include but are not limited to ipilimumab (MDX-010; Yervoy®), tremelimumab (CP-675,206; Imjudo®), botensilimab (AGEN1181), zalifrelimab (AGEN1884), BMS-188667, nurulimab (BCD-145), ADG116, AGEN2373, BA3071, BMS-986218, IBI310, JK08, ONC-392, and RGEN4659.

Examples of other CTLA-4 inhibitors which may be used in the combination treatments of the present disclosure include but are not limited to those described in U.S. Pat. Nos. 11,208,483; 11,186,840; 11,098,122; 11,078,281; 11,033,622; 10,954,301; 10,768,170; 10,711,272; 10,533,179; 10,414,814; 9,963,508; 9,834,589; 9,714,290; 9,573,999; 8,883,984; 8,784, 815; 8,697,845; 8,491,815; 8, 697,845; 8,491,895; 8,475,790; 8,318,916; 8,263,073; 8,143,379; 8,142,778; 8,017,114; 7,892,827; 7,824,679; 7,723,299; 7,605,238; 7,592,007; 7,411,047; 7,132,281; and 7,109,003.

Non-limiting examples of bispecific antibodies or antibody constructs which target both CTLA-4 and PD-1 which may be used herein include but are not limited to cadonilimab (AK104), lorigerlimab (MGD019), erfronrilimab (KN046), BCD-217, and SI-B003.

In certain embodiments of the present disclosure, the CGRP inhibitor+PD-1 antagonist includes the combination of Erenumab+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Eptinezumab+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Fremanezumab+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Galcanezumab+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Atogepant+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Olcegepant+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Obrogepant+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Rimegepant+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Avitriptan+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Eletriptan+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Naratriptan+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Rizatriptan+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Telcacepant+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Ubrogepant+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Sumatriptan+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of Zolmitriptan+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of BIBN4096+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of BI44370+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of BMS-694153+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+PD-1 antagonist includes the combination of MK-3207+pembrolizumab, or +nivolumab, or +pidilizumab, or +toripalimab, or +balstilimab, or +cemiplimab, or +dostarlimab, or +spartalizumab, or +camrelizumab, or +sintilimab, or +tislelizumab, or +durvalumab, or +avelumab, or +atezolizumab, or +AMP-224, or +MEDI0680, or +BCD100, or +BMS-936559, or +INCMGA00012, or +PDR-001, or +REGN2810, or +SAR-439684, or +SHR-1210, or +CA-170, or +STI-1110, or +STI-A1010, or +STI-A1011, or +STI-A1012, or +STI-A1013, or +STI-A1014, or +STI-A1015, or +AP-106, or +AP-105, or +AUNP-12, or +BMS-pep57, or +BMS-8, or +BMS-57, or +BMS-71, or +BMS-99, or +BMS-103, or +BMS-142, or +BMS-200, or +BMS-202, or +BMS-242, or +BMS-1001, or +BMS-1166, or +MSB-2311, or +CBT-501, or +AK-105, or +IO-102, or +IO-103, or +CX-072, or +SHR-1316, or +JTX-4014, or +GNS-1480, or +BGB-A317, or +XmAb-20717, or +FS-118, or +PSI-001, or +SN-PDL01, or +SN-PD07, or +PRS-332, or +FPT-155, or +TSR-042, or +REGN-1979, or +FAZ-053, or +MGA-012, or +MGD-013, or +M-7824, or +AK-112, or +AK-106, or +AK-104, or +AK-103, or +BI-754091, or +ENUM-244C8, or +MCLA-145, or +MCLA-134, or +IBI-308, or +WBP-3155, or +JNJ-63723283, or +SSI-361, or +CBT-502, or +PF-06801591, or +LY-3300054, or +KY-1003, or +STI-1014, or +GLS-010, or +AM-0001, or +GX-P2, or +KD-033, or +mDX-400, or +ALN-PDL, or +GB-226, or +INDUS-903, or +AMP-224, or +GX-D1.

In certain embodiments of the present disclosure, the CGRP inhibitor+CTLA-4 inhibitor includes the combination of Erenumab+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Eptinezumab+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Fremanezumab+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Galcanezumab+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Atogepant+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Olcegepant+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Obrogepant+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Rimegepant+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Avitriptan+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Eletriptan+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Naratriptan+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Rizatriptan+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Telcacepant+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Ubrogepant+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Sumatriptan+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of Zolmitriptan+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of BIBN4096+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of BI44370+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of BMS-694153+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+CTLA-4 inhibitor includes the combination of MK-3207+ipilimumab, or +tremelimumab, or +botensilimab, or +nurulimab, or +zalifrelimab, or +BMS-188667, or +ADG116, or +AGEN2373, or +BA3071, or +BMS-986218, or +IBI310, or +JK08, or +ONC-392, or +RGEN4659.

In certain embodiments of the present disclosure, the CGRP inhibitor+anti-CTLA-4/PD-1 bispecific antibody construct includes the combination of Erenumab+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Eptinezumab+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Fremanezumab+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Galcanezumab+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Atogepant+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Olcegepant+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Obrogepant+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Rimegepant+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Avitriptan+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Eletriptan+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Naratriptan+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Rizatriptan+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Telcacepant+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Ubrogepant+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Sumatriptan+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of Zolmitriptan+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of BIBN4096+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of BI44370+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of BMS-694153+cadonilimab, or +lorigerlimab, or +erfronrilimab, or +BCD-217, or +SI-B003.

In certain embodiments of the present disclosure, the CGRP inhibitor+anti-CTLA-4/PD-1 bispecific antibody construct includes the combination of MK-3207+cadonilimab, or +lorigerlimab, or +erfronrilimab, or +BCD-217, or +SI-B003.

Having generally described embodiments drawn to anti-cancer treatments in which an inhibitor of CGRP, and optionally one or more inhibitors of checkpoint proteins such as PD-1, PD-1L, or CTLA-4 is administered, a further understanding of the compositions and methods of the disclosure can be obtained by reference to certain specific experiments and examples which are provided below for purposes of illustration only and are not intended to be limiting.

### EXPERIMENTAL

### Example 1

### Inhibition of CGRP Signaling Inhibits Tumor Growth

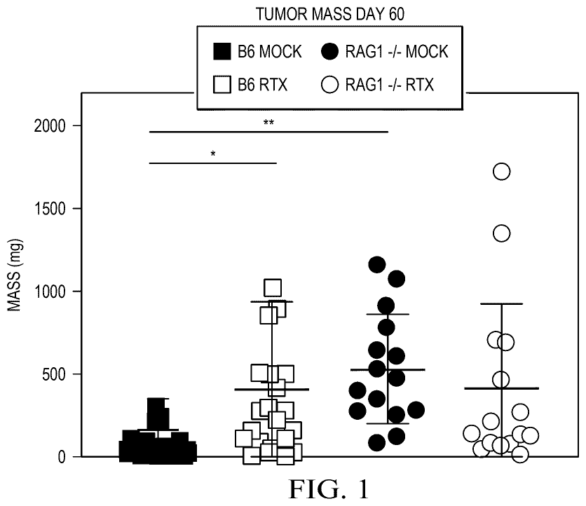

As explained above, solid tumors recruit nerve fibers, which grow in from surrounding tissue and can be pro- or anti-tumorigenic depending on the tumor and nerve type. In breast cancer for example, increased innervation correlates with poor prognosis. The impact of tumor innervation on anti-tumor immunity has been uninvestigated prior to the present work although immune cells do express receptors for numerous neurotransmitters, including the neuropeptides substance P (SP) and CGRP produced by sensory nerves. Depletion of a subset of sensory nerve fibers expressing the capsaicin receptor TRPV1 enhances tumor growth (FIG. 1). This acceleration was not observed in Rag1-deficient animals (FIG. 1), suggesting these nerve fibers act on adaptive immunity to facilitate better tumor control.

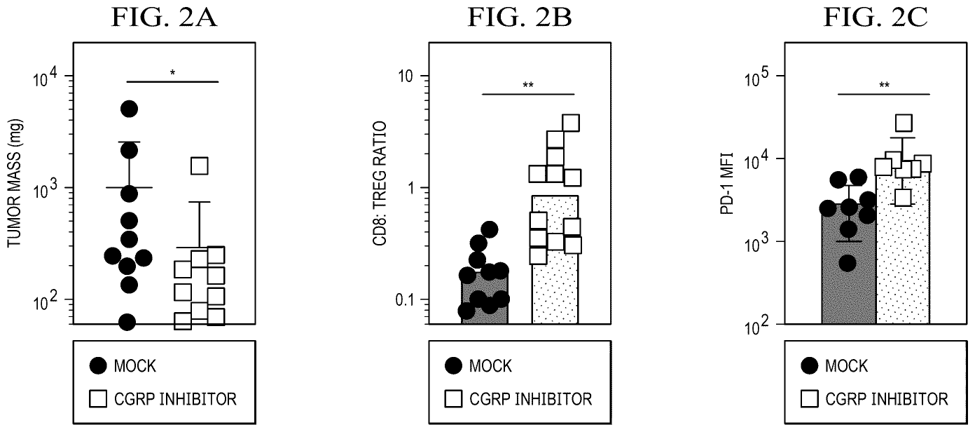

Results provided herein show that inhibiting CGRP signaling stalls tumor growth, and enhances the effector CD8⁺ T cell:Treg cell ratio in the tumor (FIGS. 2A-2B). Furthermore, effector CD8+ T cells derived from these tumors express higher levels of the inhibitory receptor PD-1 (FIG. 2C), indicating blockade of CGRP can synergize with conventional anti-PD-1 therapy to promote immune-mediated control of tumors (as further demonstrated below in Example 2).

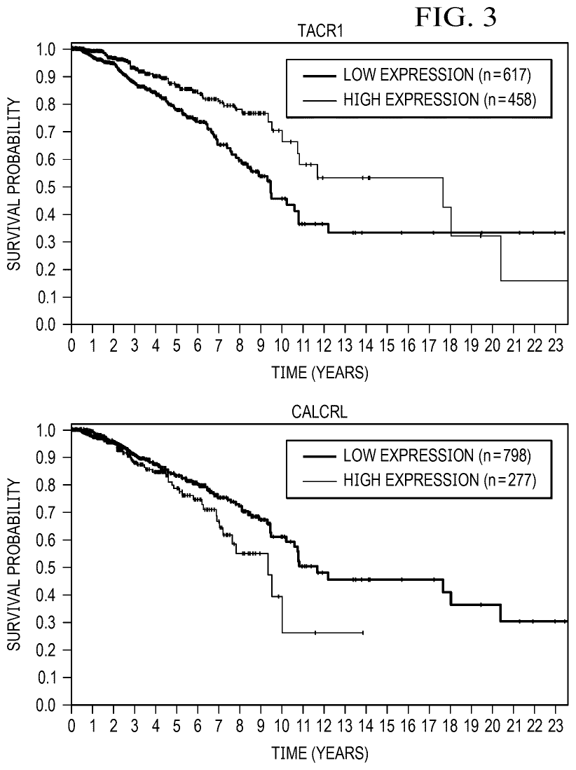

Patients with high expression of the tacykinin 1 receptor (TACR1), which is the receptor for SP, appear to have enhanced survival in breast cancer data mined from the human protein atlas (FIG. 3). In contrast, breast cancer patients with high expression of one component of the CGRP receptor, calcitonin like receptor (CALCRL) have a lower survival probability (FIG. 3). Neither receptor has previously been listed as a prognostic marker based on the data available in the human protein atlas. Nevertheless, this indicates that CGRP is an important signaling molecule for tumors, and that inhibition of CGRP signaling can be used therapeutically to inhibit tumor growth and improve infiltration of immune cells into cancerous tumors.

### Example 2

Treatment with a combination of a CGRP inhibitor and PD-1 antagonist combination inhibits tumor growth and extends survival.

### I. Anti-CGRP/Anti-PD-1 Combination Treatment Effect on Tumor Volume and Tumor Regression

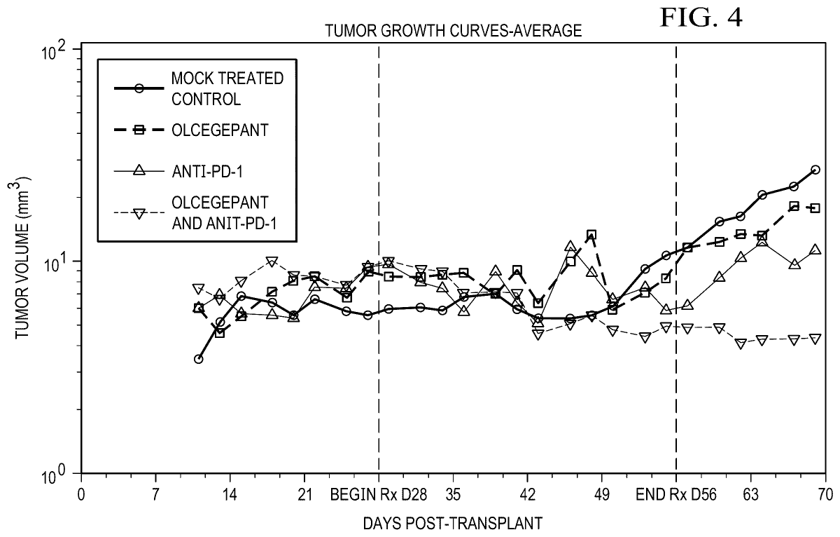

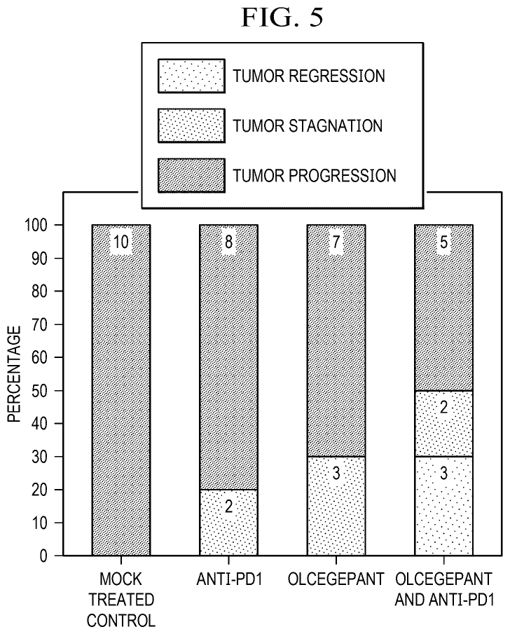

Mice were transplanted with Py230 murine mammary tumors in the mammary fat pad. 28 days after tumor implantation the drug treatments were initiated. Treatments were: (1) Mock—isotype control and vehicle control, (2) CGRP inhibitor+isotype control, (3) anti-PD-1+vehicle control, and (4) Combination: CGRP inhibitor+anti-PD-1. Treatments were terminated 56 days after transplantation. The anti-PD-1 compound was a rat IgG2a anti-mouse PD-1 antibody, clone RMP1-14, obtained from Bio X Cell, the CGRP inhibitor was olcegepant, and the isotype control was a rat IgG2a, clone 2A3, raised against human CD25, obtained from Bio X Cell. Tumor volume, as determined by caliper measurement, was measured every other day until day 70 post-transplant. Average tumor mass of 10 individuals across two experiments is depicted (FIG. 4). The results (FIG. 5) also indicated the numbers of mice in the 4 treatment groups that experienced either tumor progression (i.e., continual growth of tumor), tumor stagnation (cessation of growth for at least 3 weeks), or tumor regression (tumor no longer palpable) in each cohort. The data are composites of 2 experimental cohorts with 10 mice in each treatment group. Tumor progression occurred in all mock-treated mice. In the anti-PD-1 and anti-CGRP treatments, most tumors continued to progress except stagnation was seen in 20% and 30% of the mice tumors, respectively. In the combination treatment however, only 50% of tumors progressed, while 20% remained stagnant and 30% regressed. These results demonstrate that co-treatment of established tumors with PD-1 and CGRP inhibitors drives tumor volume regression and durable tumor rejection. The results also demonstrate a synergistic effect in the combination treatment, where 30% of tumors regressed, because no regression was seen in any of the single treatment mice tumors. If the effect was additive, the results at best would have shown 50% progression and 50% stagnation (20%+30%), with 0% regression.

### II. Anti-CGRP/Anti-PD-1 Combination Treatment Effect on Survival, Tumor Mass, and Metastasis.

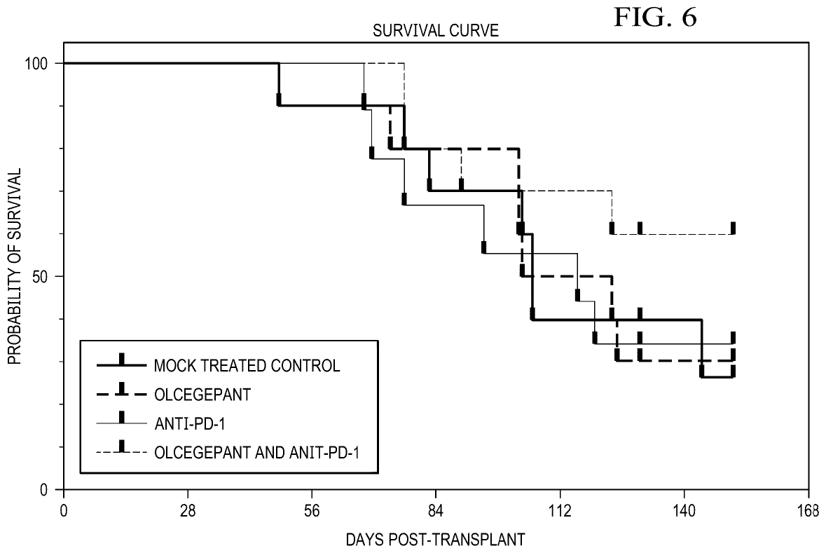

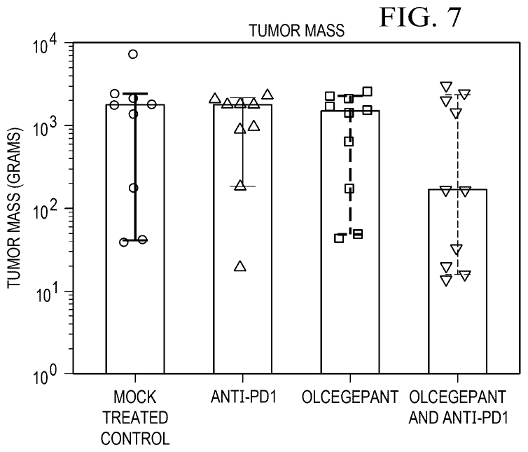

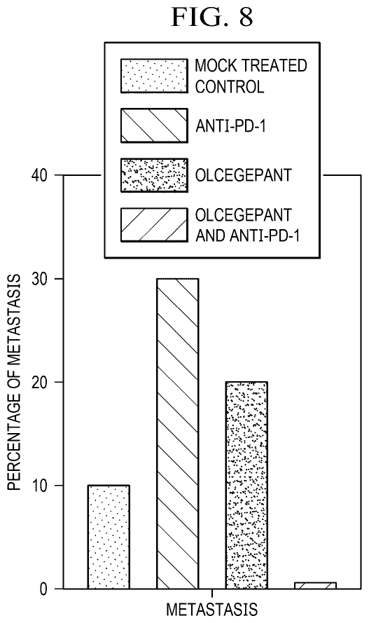

As above, mice were transplanted with Py230 tumors in the mammary fat pad. 28 days after tumor implantation the drug treatments were initiated. Treatments were: (1) Mock—isotype control and vehicle control, (2) CGRP inhibitor+isotype control, (3) anti-PD-1+vehicle control, and (4) Combination: CGRP inhibitor+anti-PD-1. The anti-PD-1 compound was a rat IgG2a anti-mouse PD-1 antibody, clone RMP1-14, obtained from Bio X Cell, the CGRP inhibitor was olcegepant, and the isotype control was a rat IgG2a, clone 2A3, raised against human CD25, obtained from Bio X Cell. Treatments were terminated 56 days after transplantation. Tumors were allowed to grow with no experimental intervention from day 56 to day 150 post-transplant. Mice were euthanized when tumors reached 15 mm in any direction. Animals found dead in the cage were evaluated for tumor metastasis to lungs. Results are a composite of 2 experimental cohorts, n=10 mice for each treatment group. The overall survival is depicted in FIG. 6. The results demonstrate that the anti-CGRP/anti-PD-1 combination treatment in mice with established tumors increases animal survival. After about 150 days, approximately 60% of the mice treated with the drug combination were still alive, whereas in the control or single-treatment groups, survival ranged from about 26% to slightly over 30%. Thus, survival rates approximately doubled with the combination treatment. Primary tumor mass in mice was determined at 150 days post-transplant. Primary tumor mass was, on average, much lower in the combination treatment mice than in the control or single treatment mice (FIG. 7). Mice were then analyzed for tumor metastases in the lungs. FIG. 8 shows the percentages of mice with visible lung metastases at death (earlier than day 150 post-transplant) or at the end of the course of treatment. Lungs in tumor-established mice exposed to the anti-PD-1/anti-CGRP combination treatment showed almost no lung metastases, whereas 10-30% of the lungs of control and single treatment mice showed metastases. These results demonstrate that combination treatment of mice with established breast tumors limits tumor mass and prevents development of lung metastases.

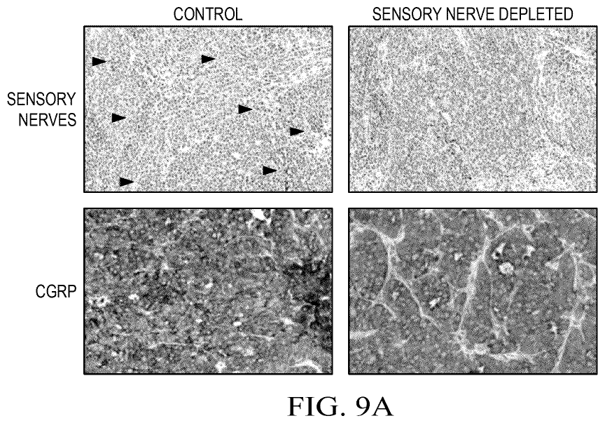

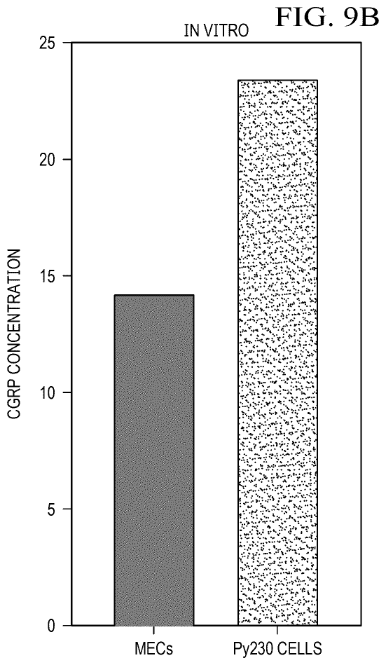

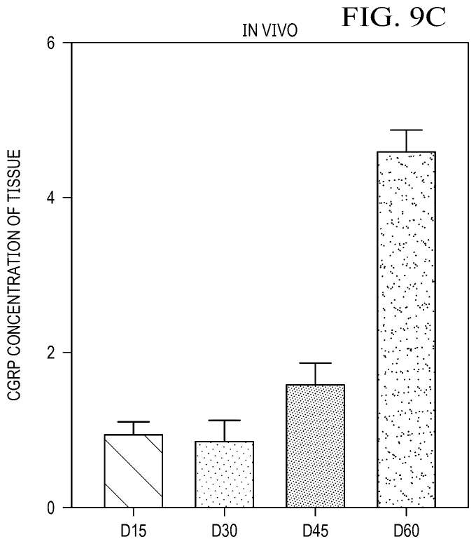

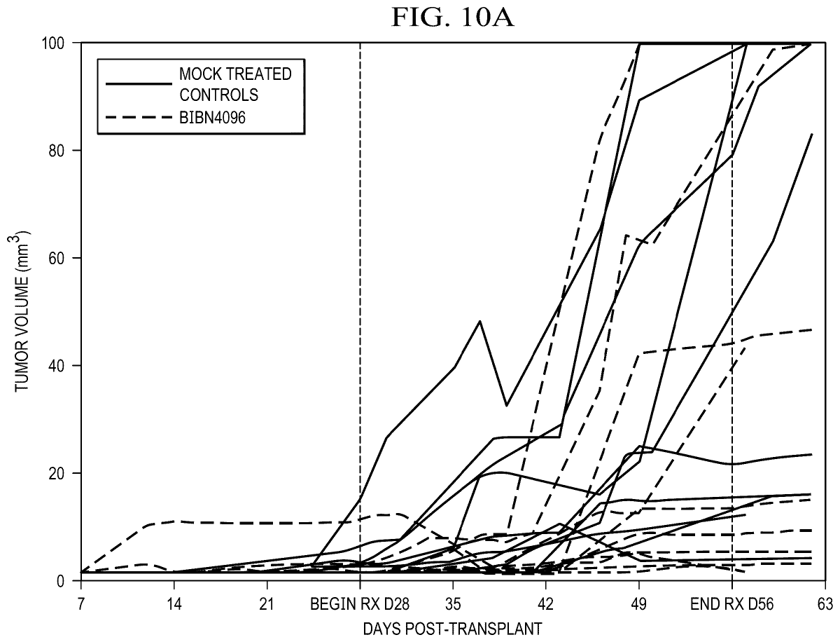

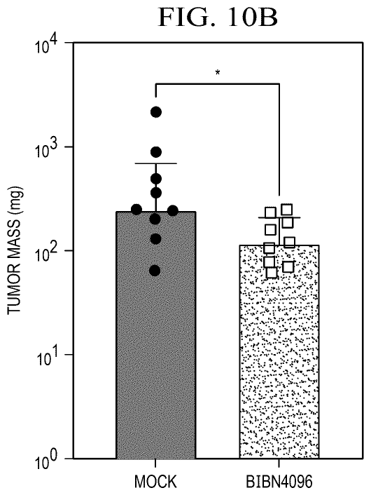

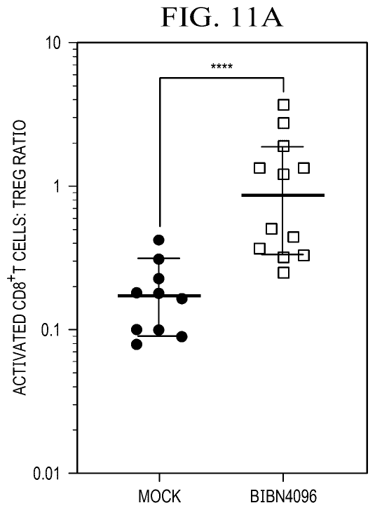

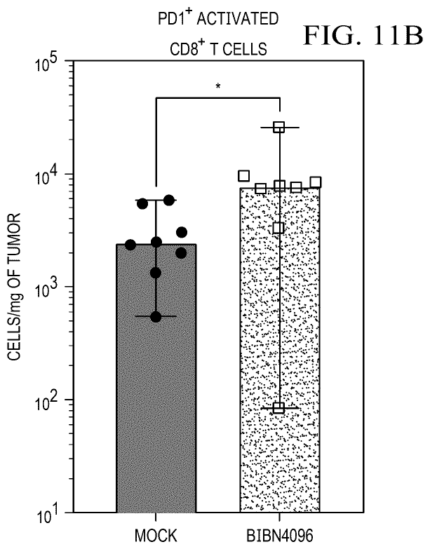

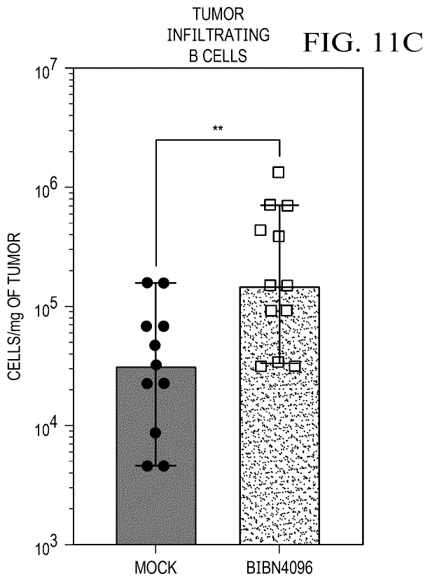

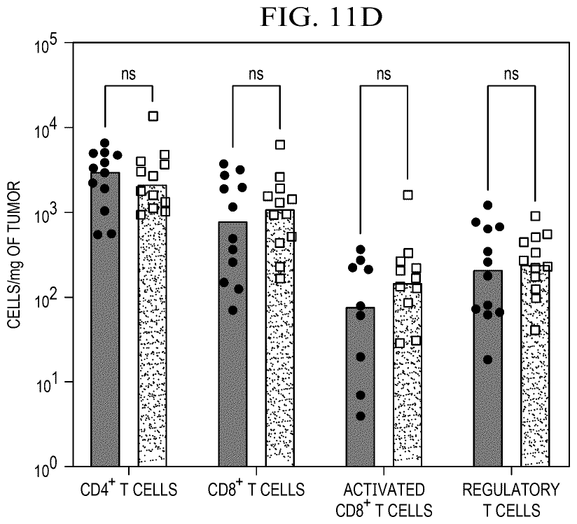

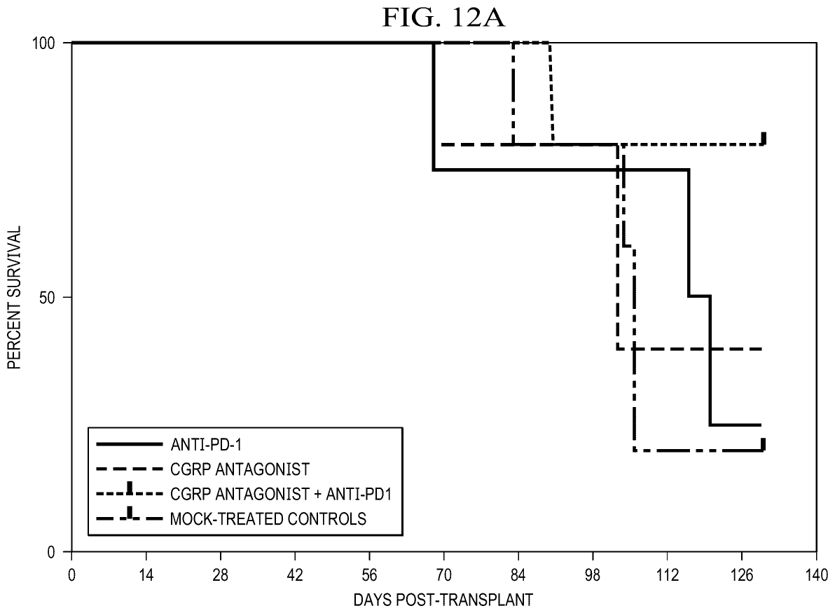

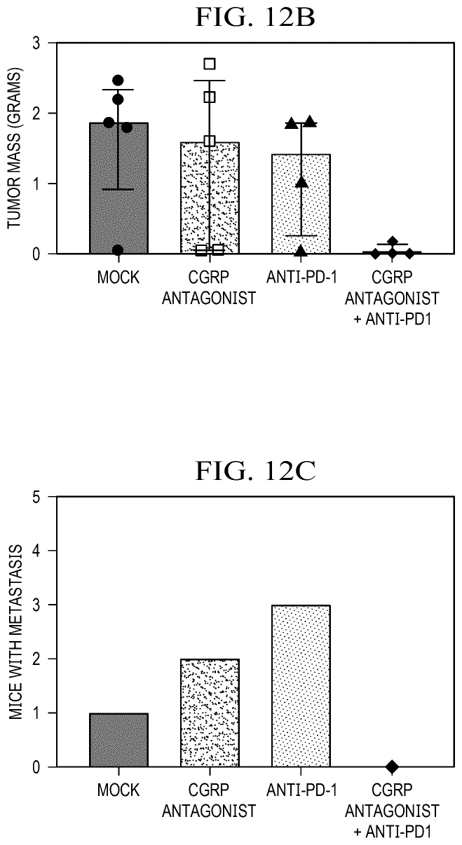

Further results which elucidate the contribution of tumor derived CGRP in tumor growth and immune evasion are shown in FIGS. 9-12. FIGS. 9A, 9B and 9C show that breast cancer produces CGRP in the absence of sensory nerves. FIGS. 10A and 10B show that BIBN4096 (a.k.a., Olcegepant), a CGRP antagonist, stops growth of triple negative breast cancer and decreases tumor mass in vivo. FIGS. 11A, 11B, 11C and 11D show that BIBN4096 enhances CD8+ T cell activation and tumor infiltrating B cells. BIBN4096-treated mice have a higher ratio of activated CD8+ T cells to regulatory T cells (T reg) compared to the mock treated controls. Activated CD8+ T cells have an increased expression of the inhibitory receptor PD-1 in BIBN4096-treated mice compared to their controls. FIGS. 12A, 12B and 12C show that BIBN4096 combined with PD-1 receptor blockade causes regression of triple negative breast cancer tumors. Kaplan Meier survival graphs show that mice co-treated with BIBN4096 and anti-PD-1 antibody have better survival compared to their respective controls (FIG. 12A). Cotreatment with BIBN4096 and anti-PD-1 antibody reduces treatment mass (FIG. 12B) and tumor metastases (FIG. 12C).

As is demonstrated above, in particular embodiments, the present disclosure is directed to a method of treating a solid tumor cancer in a subject in need of such treatment, wherein the method comprises administering to the subject a calcitonin gene-related peptide (CGRP) inhibitor, wherein the treatment reduces the growth, volume, or mass of the solid tumor. The CGRP inhibitor may be an antibody or a binding fragment thereof, or a small molecule, which binds to CGRP or to CGRP receptor thereby inhibiting CGRP from binding to CGRP receptor. The CGRP inhibitor may be selected, for example, from eptinezumab, fremanezumab, galcanezumab, erenumab, telcagepant, atogepant, ubrogepant, olcegepant (BIBN4096), rimegepant, vazegepant, obrogepant, avitriptan, eletriptan, naratriptan, rizatriptan, sumatriptan, zolmitriptan, BI44370, BMS 694153, and MK-3207. The treatment may further comprise a step of administering at least one checkpoint inhibitor to the subject. The at least one checkpoint inhibitor may be selected from, for example, a Programmed Cell Death Protein-1 (PD-1) antagonist, a Cytotoxic T-lymphocyte-Associated protein 4 (CTLA-4) inhibitor, and an anti-PD-1/anti-CTLA-4 bispecific antibody construct. The PD-1 antagonist may be selected from, for example, pembrolizumab, nivolumab, pidilizumab, toripalimab, balstilimab, cemiplimab, dostarlimab, spartalizumab, camrelizumab, sintilimab, tislelizumab, durvalumab, avelumab, atezolizumab, AMP-224, MEDI0680, BCD100, BMS-936559, INCMGA00012, PDR-001, REGN2810, SAR-439684, SHR-1210, CA-170, STI-1110, STI-A1010, STI-A1011, STI-A1012, STI-A1013, STI-A1014, STI-A1015, AP-106, AP-105, AUNP-12, BMS-pep57, BMS-8, BMS-57, BMS-71, BMS-99, BMS-103, BMS-142, BMS-200, BMS-202, BMS-242, BMS-1001, BMS-1166, MSB-2311, CBT-501, AK-105, IO-102, IO-103, CX-072, SHR-1316, JTX-4014, GNS-1480, BGB-A317, XmAb-20717, FS-118, PSI-001, SN-PDL01, SN-PD07, PRS-332, FPT-155, TSR-042, REGN-1979, FAZ-053, MGA-012, MGD-013, M-7824, AK-112, AK-106, AK-104, AK-103, BI-754091, ENUM-244C8, MCLA-145, MCLA-134, IBI-308, WBP-3155, JNJ-63723283, SSI-361, CBT-502, PF-06801591, LY-3300054, KY-1003, STI-1014, GLS-010, AM-0001, GX-P2, KD-033, mDX-400, ALN-PDL, GB-226, INDUS-903, AMP-224, and GX-D1. The CTLA-4 inhibitor may be an anti-CTLA-4 antibody, such as, for example, ipilimumab, tremelimumab, botensilimab, nurulimab, zalifrelimab, BMS-188667, ADG116, AGEN2373, BA3071, BMS-986218, IBI310, JK08, ONC-392, and RGEN4659, and binding fragments thereof. The anti-PD-1/anti-CTLA-4 bispecific antibody may be selected from, for example, cadonilimab, lorigerlimab, erfronrilimab, BCD-217, and SI-B003, and binding fragments thereof. The cancer may be one or more of breast cancer, triple negative breast cancer, melanoma, bladder cancer, esophageal cancer, pancreatic cancer, gastric cancer, non-small-cell lung cancer (NSCLC), renal cell carcinoma, Hodgkin's lymphoma, Non-Hodgkin's lymphoma (NHL), endometrial cancer, squamous cell cancer of the head and neck (SCCHN), colorectal cancer, a glioblastoma, ovarian cancer, pharyngeal cancer, prostate cancer, a small round blue cell tumor, small cell lung cancer, hepatocellular carcinoma, ovarian cancer, urothelial cancer, colorectal cancer, lymphoma, a neuroblastoma, a rhabdomyosarcoma, leiomyosarcoma, liposarcoma, synovial sarcoma, malignant peripheral sheath tumor (MPNST), and multiple myeloma (MM). The antibody or binding fragment thereof may be a human antibody, a mouse antibody, a chimeric antibody, a humanized antibody, or a chimeric humanized antibody, for example. The CGRP inhibitor and/or at least one checkpoint inhibitor may be administered to the subject in an amount of, for example, about 0.1 mg/kg to about 50 mg/kg. The CGRP inhibitor and the at least one checkpoint inhibitor, when administered in combination, may be administered to the subject simultaneously, concurrently, sequentially, successively, alternately, or separately. The CGRP inhibitor and the at least one checkpoint inhibitor may interact synergistically to reduce the volume of the solid tumor. The CGRP inhibitor and the at least one checkpoint inhibitor may be coadministered to the subject with an anti-cancer drug. The treatment may enhance (increase) immune cell infiltration into the solid tumor. The immune cells having enhanced infiltration into the solid tumor may comprise activated CD8+ T cells.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the embodiments described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of methods and procedures as well as of the principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in exemplary claims herein below, it is not intended that the present disclosure be limited to these particular exemplary claims.

What is claimed is:

1. A method of treating a solid tumor cancer in a subject in need of such treatment, comprising administering to the subject a calcitonin gene-related peptide (CGRP) inhibitor, wherein the treatment reduces the growth, volume, or mass of the solid tumor.

2. The method of claim 1, wherein the CGRP inhibitor is an antibody or a binding fragment thereof, or a small molecule, which binds to CGRP or to CGRP receptor thereby inhibiting CGRP from binding to CGRP receptor.

3. The method of claim 1, wherein the CGRP inhibitor is selected from eptinezumab, fremanezumab, galcanezumab, erenumab, olcegepant (BIBN4096), telcagepant, atogepant, ubrogepant, rimegepant, vazegepant, obrogepant, avitriptan, eletriptan, naratriptan, rizatriptan, sumatriptan, zolmitriptan, BI44370, BMS 694153, and MK-3207.

4. The method of claim 1, wherein the treatment further comprises administering at least one checkpoint inhibitor to the subject.

5. The method of claim 4, wherein the at least one checkpoint inhibitor is selected from the group consisting of a Programmed Cell Death Protein-1 (PD-1) antagonist, a Cytotoxic T-lymphocyte-Associated protein 4 (CTLA-4) inhibitor, and an anti-PD-1/anti-CTLA-4 bispecific antibody construct.

6. The method of claim 5, wherein the PD-1 antagonist is selected from the group consisting of pembrolizumab, nivolumab, pidilizumab, toripalimab, balstilimab, cemiplimab, dostarlimab, spartalizumab, camrelizumab, sintilimab, tislelizumab, durvalumab, avelumab, atezolizumab, AMP-224, MEDI0680, BCD100, BMS-936559, INCMGA00012, PDR-001, REGN2810, SAR-439684, SHR-1210, CA-170, STI-1110, STI-A1010, STI-A1011, STI-A1012, STI-A1013, STI-A1014, STI-A1015, AP-106, AP-105, AUNP-12, BMS-pep57, BMS-8, BMS-57, BMS-71, BMS-99, BMS-103, BMS-142, BMS-200, BMS-202, BMS-242, BMS-1001, BMS-1166, MSB-2311, CBT-501, AK-105, IO-102, IO-103, CX-072, SHR-1316, JTX-4014, GNS-1480, BGB-A317, XmAb-20717, FS-118, PSI-001, SN-PDL01, SN-PD07, PRS-332, FPT-155, TSR-042, REGN-1979, FAZ-053, MGA-012, MGD-013, M-7824, AK-112, AK-106, AK-104, AK-103, BI-754091, ENUM-244C8, MCLA-145, MCLA-134, IBI-308, WBP-3155, JNJ-63723283, SSI-361, CBT-502, PF-06801591, LY-3300054, KY-1003, STI-1014, GLS-010, AM-0001, GX-P2, KD-033, mDX-400, ALN-PDL, GB-226, INDUS-903, AMP-224, and GX-D1.

7. The method of claim 5, wherein the CTLA-4 inhibitor is an anti-CTLA-4 antibody selected from ipilimumab, tremelimumab, botensilimab, nurulimab, zalifrelimab, BMS-188667, ADG116, AGEN2373, BA3071, BMS-986218, IBI310, JK08, ONC-392, and RGEN4659, and binding fragments thereof.

8. The method of claim 5, wherein the anti-PD-1/anti-CTLA-4 bispecific antibody is selected from the group consisting of cadonilimab, lorigerlimab, erfronrilimab, BCD-217, and SI-B003, and binding fragments thereof.

9. The method of claim 1, wherein the solid tumor cancer is selected from breast cancer, triple negative breast cancer, melanoma, bladder cancer, esophageal cancer, pancreatic cancer, gastric cancer, non-small-cell lung cancer (NSCLC), renal cell carcinoma, Hodgkin's lymphoma, Non-Hodgkin's lymphoma (NHL), endometrial cancer, squamous cell cancer of the head and neck (SCCHN), colorectal cancer, a glioblastoma, ovarian cancer, pharyngeal cancer, prostate cancer, a small round blue cell tumor, small cell lung cancer, hepatocellular carcinoma, ovarian cancer, urothelial cancer, colorectal cancer, lymphoma, a neuroblastoma, a rhabdomyosarcoma, leiomyosarcoma, liposarcoma, synovial sarcoma, malignant peripheral sheath tumor (MPNST), and multiple myeloma (MM).

10. The method of claim 2, wherein the antibody or binding fragment thereof is selected from the group consisting of a human antibody, a mouse antibody, a chimeric antibody, a humanized antibody, and a chimeric humanized antibody.

11. The method of claim 4, wherein the CGRP inhibitor and/or at least one checkpoint inhibitor is administered to the subject in an amount of about 0.1 mg/kg to about 50 mg/kg.

12. The method of claim 4, wherein the CGRP inhibitor and the at least one checkpoint inhibitor are administered to the subject simultaneously, concurrently, sequentially, successively, alternately, or separately.

13. The method of claim 4, wherein the CGRP inhibitor and the at least one checkpoint inhibitor interact synergistically to reduce the volume of the solid tumor.

14. The method of claim 4, wherein the CGRP inhibitor and the at least one checkpoint inhibitor are coadministered to the subject with an anti-cancer drug.

15. The method of claim 1, wherein the treatment enhances immune cell infiltration into the solid tumor.

16. The method of claim 15, wherein the immune cells having enhanced infiltration into the solid tumor comprise activated CD8+ T cells.

17. A method of treating a solid tumor cancer in a subject in need of such treatment, comprising administering to the subject a calcitonin gene-related peptide (CGRP) inhibitor and a checkpoint inhibitor, wherein the treatment reduces the growth, volume, or mass of the solid tumor.

18. The method of claim 17, wherein the CGRP inhibitor is selected from eptinezumab, fremanezumab, galcanezumab, erenumab, olcegepant (BIBN4096), telcagepant, atogepant, ubrogepant, rimegepant, vazegepant, obrogepant, avitriptan, eletriptan, naratriptan, rizatriptan, sumatriptan, zolmitriptan, BI44370, BMS 694153, and MK-3207; and the checkpoint inhibitor is selected from the group consisting of a Programmed Cell Death Protein-1 (PD-1) antagonist, a Cytotoxic T-lymphocyte-Associated protein 4 (CTLA-4) inhibitor, and an anti-PD-1/anti-CTLA-4 bispecific antibody construct.

19. The method of claim 18, wherein the PD-1 antagonist is selected from the group consisting of pembrolizumab, nivolumab, pidilizumab, toripalimab, balstilimab, cemiplimab, dostarlimab, spartalizumab, camrelizumab, sintilimab, tislelizumab, durvalumab, avelumab, atezolizumab, AMP-224, MEDI0680, BCD100, BMS-936559, INCMGA00012, PDR-001, REGN2810, SAR-439684, SHR-1210, CA-170, STI-1110, STI-A1010, STI-A1011, STI-A1012, STI-A1013, STI-A1014, STI-A1015, AP-106, AP-105, AUNP-12, BMS-pep57, BMS-8, BMS-57, BMS-71, BMS-99, BMS-103, BMS-142, BMS-200, BMS-202, BMS-242, BMS-1001, BMS-1166, MSB-2311, CBT-501, AK-105, IO-102, IO-103, CX-072, SHR-1316, JTX-4014, GNS-1480, BGB-A317, XmAb-20717, FS-118, PSI-001, SN-PDL01, SN-PD07, PRS-332, FPT-155, TSR-042, REGN-1979, FAZ-053, MGA-012, MGD-013, M-7824, AK-112, AK-106, AK-104, AK-103, BI-754091, ENUM-244C8, MCLA-145, MCLA-134, IBI-308, WBP-3155, JNJ-63723283, SSI-361, CBT-502, PF-06801591, LY-3300054, KY-1003, STI-1014, GLS-010, AM-0001, GX-P2, KD-033, mDX-400, ALN-PDL, GB-226, INDUS-903, AMP-224, and GX-D1; the CTLA-4 inhibitor is an anti-CTLA-4 antibody selected from ipilimumab, tremelimumab, botensilimab, nurulimab, zalifrelimab, BMS-188667, ADG116, AGEN2373, BA3071, BMS-986218, IBI310, JK08, ONC-392, and RGEN4659, and binding fragments thereof, and the anti-PD-1/anti-CTLA-4 bispecific antibody is selected from the group consisting of cadonilimab, lorigerlimab, erfronrilimab, BCD-217, and SI-B003, and binding fragments thereof.

20. The method of claim 17, wherein the solid tumor cancer is selected from breast cancer, triple negative breast cancer, melanoma, bladder cancer, esophageal cancer, pancreatic cancer, gastric cancer, non-small-cell lung cancer (NSCLC), renal cell carcinoma, Hodgkin's lymphoma, Non-Hodgkin's lymphoma (NHL), endometrial cancer, squamous cell cancer of the head and neck (SCCHN), colorectal cancer, a glioblastoma, ovarian cancer, pharyngeal cancer, prostate cancer, a small round blue cell tumor, small cell lung cancer, hepatocellular carcinoma, ovarian cancer, urothelial cancer, colorectal cancer, lymphoma, a neuroblastoma, a rhabdomyosarcoma, leiomyosarcoma, liposarcoma, synovial sarcoma, malignant peripheral sheath tumor (MPNST), and multiple myeloma (MM).
